(19) United States
(12) Patent Application Publication
(10) Pub. No.: US 2005/0220781 A1
(43) Pub. Date: Oct. 6, 2005

(54) IFIX, a novel HIN-200 protein, for cancer therapy

(76) Inventors: Duen-Hwa Yan, Houston, TX (US); Yi Ding, Pearland, TX (US); Li Wang, Pearland, TX (US); Mien-Chie Hung, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/934,861

(22) Filed: Sep. 3, 2004

Related U.S. Application Data
(60) Provisional application No. 60/500,191, filed on Sep. 4, 2003.
(60) Provisional application No. 60/551,511, filed on Mar. 9, 2004.

(51) Int. Cl. A61K 38/48; C07H 21/04; C12N 9/64; C12N 15/09

(52) U.S. Cl. 424/94.63; 514/12; 530/350; 536/23.5; 435/69.1; 435/320.1; 435/325; 435/226

(57) ABSTRACT

The present invention regards IFIX proteins, polypeptides, peptides, and the polynucleotides that encode them. In particular embodiments, the IFIX proteins, polypeptides, and/or peptides comprise tumor suppressive, anti-cell proliferative pro-apoptotic and/or cell cycle arrest-inducing activities. In more particular embodiments, these forms are useful for cancer therapy, particularly when administered in combination with liposomes.

This application claims priority to U.S. Provisional Patent Application Ser. No. 60/500,191, filed Sep. 4, 2003 and to U.S. Provisional Patent Application Ser. No. 60/551,511, filed Mar. 9, 2004, both of which are incorporated by reference herein in their entirety.

The present invention was generated at least in part by grants from the Department of Defense (DAMD17-99-1-9270 and DAMD17-02-1-0451) and Texas Advanced Technology Program under Grant No. 003657-0082-1999. The United States Government may have certain rights in the invention.

### FIELD OF THE INVENTION

The present invention is directed to the fields of cell biology, molecular biology, cancer biology, and medicine. More particularly, the present invention regards IFIX sequences and methods of using same that are useful, in some embodiments, for cancer therapy.

### BACKGROUND OF THE INVENTION

The IFN family of cytokines is known for its growth inhibitory activity (Pestka et al., 1987), which plays an important role in IFN-mediated anti-tumor activity (Brenning et al., 1985; Kimchji et al., 1988). IFN-inducible proteins are thought to mediate the anti-tumor activity (Lengyel, 1993). HIN-200 family proteins are IFN-inducible proteins that share a signature 200-amino acid motif of type a and/or b. Three human (IFI16, MNDA, and AIM2) and five mouse (p202a, p202b, p203, p204, and D3) HIN-200 family proteins have been identified (Lengyel et al., 1995; Landolfo et al., 1998; Johnstone and Trapani, 1999; Choubey, 2000). Genes encoding HIN-200 family proteins in both mouse and human are located at chromosome 1q21-23 and form a gene cluster (Lengyel et al., 1995; Johnstone and Trapani, 1999). HIN-200 proteins are primarily nuclear proteins involved in transcriptional regulation of genes important for cell cycle control, differentiation, and apoptosis (Lengyel et al., 1995; Johnstone and Trapani, 1999; Choubey, 2000). The anti-tumor activity of a HIN-200 protein has been demonstrated. Previously, the inventors have shown that p202a suppressed tumor growth, reduced tumorigenicity, induced apoptosis, and suppressed metastasis and tumor angiogenesis of many human cancer cell lines (Yan et al., 1999; Wen et al., 2001; Wen et al., 2000; Ding er al,. 2002). The amino acid sequence identity between the three human HIN-200 family proteins and the mouse p202a are 40% or less, thus none of these human proteins appears to be the ortholog of p202a.

### BRIEF SUMMARY OF THE INVENTION

The present invention provides novel therapeutic IFIX compositions and methods, particularly for cancer, and a skilled artisan recognizes any additional means in an arsenal to fight cancer is beneficial to public health.

Hematopoietic interferon (IFN)-inducible nuclear proteins with 200-amino-acid repeat (HIN-200) are proteins that contain one or two copies of signature 200-amino-acid motif and whose expressions are induced by IFN. Three human HIN-200 proteins have previously been identified. In a search for the potential human ortholog of mouse p202a, the present inventors recently identified a new member of the human HIN-200 protein family, IFIX (IFN-Inducible protein X). As shown herein, the expression of IFIX is reduced in breast tumor tissues and breast cancer cell lines, and the enforced expression of IFIX in breast cancer cell lines reduces their growth and tumorigenicity. The present inventors also demonstrate the treatment efficacy of an IFIX-based gene therapy in an orthotopic breast cancer model. Thus, IFIX functions as a tumor suppressor and is useful as a therapeutic agent in breast cancer treatment.

Specifically, the studies associated with the present invention showed that the expression of IFIX in hematopoietic cell lines is induced by IFN. Expression of IFIX reduces anchorage-dependent and -independent growth in vitro and tumorigenicity in nude mice of two breast cancer cell lines that do not express endogenous IFIX. Moreover, liposome-mediated IFIX gene transfer suppresses the growth of already-formed tumors in a breast cancer xenograft model. IFIX appears to suppress breast cancer cell growth by specifically increasing the expression of the cyclin-dependent kinase (CDK) inhibitor p21^{CIP1} in a p53-independent manner leading to the inhibition of the kinase activity of CDK2 and p34^{Cdc2}. Thus, IFIX possesses tumor suppressor activity in breast cancer and is useful as a therapeutic agent in cancer treatment.

The present invention is directed to a system and methods related IFIX, and particularly to its anti-tumor effect(s). In the present invention, the inventors demonstrate the novel finding that IFIX exerted strong antitumor activity in both in vivo and in vitro systems. The Examples presented herein indicate that the transfection with a polynucleotide encoding an IFIX polypeptide induces apoptosis in various human cancers. This provides therapeutics and methods of using same for the present invention, such as the IFIX polynucleotide in gene therapy for breast, ovarian, and prostate cancer, as well as other cancers.

Thus, the present invention generally relates to methods for inhibiting proliferation in a cancer cell and/or tumor cell, the method comprising contacting the cell with an IFIX polypeptide in an amount effective to inhibit proliferation. The IFIX polypeptide referred to herein in some embodiments has anti-cell proliferative, pro-apoptotic, and/or anti-tumor activity. Inhibition of proliferation may be indicated by an induction of apoptosis of a cell, such as, for example, in cell culture, inhibition of growth of a cancer cell line, reduction in size of a tumor, and/or an increase in survivability, in exemplary embodiments. More preferably, in some embodiments the cell in which proliferation is to be inhibited is a cell in a living organism, for example a human. The inhibition of such transformation has great utility in the prevention and/or treatment of such transformation-driven events as cancer, tumorigenesis, and/or metastasis.

In specific embodiments, IFIX functions as a tumor suppressor. Using breast cancer as a model, it was shown that IFIX expression in breast cancer cells was associated with reduced growth rate, and suppressed transformation phenotype and tumorigenicity. Interestingly, IFIX upregulates p21^{CIP1}, a CDK inhibitor, and that may be associated with IFIX-mediated tumor suppression. Furthermore, the therapeutic efficacy of IFIX treatment was demonstrated in a breast cancer xenograft model, showing the utility of using IFIX as a therapeutic gene in an exemplary pre-clinical gene therapy model. Together, the data show that IFIX functions as a tumor suppressor.

The tumor suppressor activity of IFIX is shown by determining the expression of IFIX in human breast tumor, the tumor suppressor activity of IFIX in knockdown cell model, and the tumor suppressor activity of IFIX in transgenic mice model. Furthermore, the mechanism(s) of IFIX regulation may be determined, such as by showing the transcriptional downregulation of IFIX promoter in breast cancer cells, the IFIX promoter activity between normal breast and breast cancer cells, and/or the mechanism of IFIX transcriptional downregulation in breast cancer cells. The role of p21^{CIP} in IFIX-mediated tumor suppression may be shown by demonstrating the role of p21^{CIP1} in IFIX-mediated tumor suppression, and/or the mechanism by which IFIX upregulates p21^{CIP1} transcription. Finally, the IFIX-mediated tumor suppressor activity in pre-clinical breast cancer gene therapy setting may be demonstrated, such as by determining the anti-tumor activity of IFIX in primary breast cancer tissues, the IFIX-mediated anti-tumor activity using a liposome-based gene delivery system, and/or the IFIX-mediated antitumor activity using an adenovirus-based gene delivery system.

An IFIX polypeptide may be contacted with or introduced to a cell through any of a variety of manners known to those of skill. The IFIX polypeptide may be introduced through direct introduction of an IFIX polypeptide to a cell. In this case, the IFIX polypeptide may be obtained through any method known in the art, although it is expected that in vitro production of the IFIX polypeptide in a cell culture system may be a preferred manner of obtaining IFIX. In a specific embodiment, the IFIX polypeptide comprises a protein transduction domain, such as HIV Tat. In other embodiments, an IFIX molecule comprises a nuclear localization domain.

IFIX may also be introduced to a cell via the introduction of a polynucleotide that encodes the IFIX polypeptide. For example, RNA or DNA encoding IFIX may be introduced to the cell by any manner known in the art. In certain preferred embodiments, the IFIX is introduced into the cell through the introduction of a DNA segment that encodes IFIX. In some such embodiments, it is envisioned that the DNA segment further comprises the IFIX gene (or IFIX polynucleotide) operatively linked to associated control sequences, such as its endogenous control sequence(s). For example, the IFIX gene may be operatively linked to a suitable promoter and a suitable terminator sequence. The construction of such gene/control sequence DNA constructs is well-known within the art. In particular embodiments, the promoter is selected from the group comprising mammary tumor epithelium specific promoter, such as mouse mammary tumor virus long termial repeat (MMTV); CMV; telomerase; TCF-4; VEGF; CMV-GADPH, human alpha-lactalbumin (hALA), and ovine beta-lactoglobulin promoters. In certain embodiments for introduction, the DNA segment may be located on a vector, for example, a plasmid vector or a viral vector. The virus vector may be, for example, selected from the group comprising retrovirus, adenovirus, herpesvirus, vaccina virus, and adeno-associated virus. Such a DNA segment may be used in a variety of methods related to the invention. The vector may be used to deliver an IFIX gene to a cell in one of the gene-transfer embodiments of the invention. Also, such vectors can be used to transform cultured cells, and such cultured cells could be used, inter alia, for the expression of IFIX in vitro.

The present invention is useful for all types of cancer, since IFIX, in exemplary embodiments, kills cancer cells regardless of their survival tactics adopted by many cancer cells, such as growth factor receptor and AKT pathways. In a particular embodiment, IFIX is effective on solid tumors, such as, for example, sarcoma, lung, brain, pancreatic, liver, bladder, gastrointestinal cancers, and hematologic malignancies, such as leukemia, lymphoma, and myeloma. In exemplary embodiments, the present invention is useful for cancers that are estrogen receptor positive, EGF receptor overexpressing, Her2/neu-overexpressing, Her-2/neu-nonoverexpressing, Akt overexpressing, and angrogen independent. That is, IFIX is effective on cancer cells regardless of their status of oncogene overexpression, such as Her-2/neu, EGFR, AKT, or whether their growth is hormone dependent (such as, for example, MCF-7) or not (such as, for example, PC3).

For example, contained herein are specific data showing effectiveness of IFIX against cell lines tested from breast cancer, including: MCF-10A, MCF-12A, (estrogen receptor positive), MDA-MB-468 (EGF receptor overexpressing), and MCF-7 cells. MDA-MB-468 is ER-negative but MCF-7 is ER-positive. MCF-10A, MCF-12A, MDA-MB-468, and MCF-7 are low in HER-2 levels.

In some embodiments of the present invention, IFIX is effective on cancer cells regardless of their status of p53 expression, and as such acts in an anti-tumor capacity in both a p53-dependent or a p53-independent manner. For example, the p53-dependent mechanism by which IFIX upregulates p21 comprises inhibition of HDM2, a negative regulator of p53. Inhibition of HDM2 by IFIX leads to increased levels of p53 and activation of the p53 target genes, including at least p21. In the absence of p53, such as in MD468 cells comprising mutation in p53, p21 can still be upregulated, which suggests this mechanism can also be p53-independent. Not wanting to be bound by theory, the p53-independent mechanism may utilize the degradation of HDM2 by IFIX. That is, HDM2 is able to bind to p21 and de-stabilize it. If IFIX destabilizes HDM2, then there will be fewer or no HDM2 available to de-stabilize p21.

In one embodiment of the present invention, IFIX anti-tumor activity functions through its association with HDM2. The expression of HDM2 is overexpressed in a variety of human tumors, and its gene product localizes predominantly to the nucleus, where it acts as an inhibitor of the p53 tumor suppressor gene product. As shown herein, IFIX activates p53 by downregulating HDM2, and it furthermore promotes degradation of HDM2. IFIX interacts with HDM2 in a complex, and in some embodiments the interaction comprises direct binding within the complex, whereas in other embodiments the interaction is indirect, wherein one or more molecules bridges binding of HDM2 and IFIX in the complex.

In particular embodiments, IFIX is introduced into a cell that is a human cell. In many embodiments the cell is a tumor cell. In some presently preferred embodiments the tumor cell is a breast tumor cell, a prostrate tumor cell, or an ovarian tumor cell. However, IFIX may be introduced into other cells including, but not limited to, a bladder cancer cell, a testicular cancer cell, a colon cancer cell, a skin cancer cell, a lung cancer cell, a pancreatic cancer cell, a stomach cancer cell, an esophageal cancer cell, a brain cancer cell, a leukemia cancer cell, a liver cancer cell, an endometrial cancer cell, or a head and neck cancer cell. In some embodiments, the IFIX composition is introduced by injection.

In some embodiments of the present invention, the inventors' discovery that IFIX is able to inhibit proliferation will be used in combination with other anti-transformation/anti-cancer therapies. These other therapies may be known at the time of this application, or may become apparent after the date of this application. IFIX may be used in combination with other therapeutic polypeptides, polynucleotides encoding other therapeutic polypeptides, or chemotherapeutic agents. For example, IFIX may be used in conjunction with other known polypeptides, such as TNFα or p53. IFIX may be used in conjunction with any suitable chemotherapeutic agent. In one representative embodiment, the chemotherapeutic agent is taxol. IFIX also may be used in conjunction with radiotherapy. The type of ionizing radiation constituting the radiotherapy may be selected from the group comprising x-rays, γ-rays, and microwaves. In certain embodiments, the ionizing radiation may be delivered by external beam irradiation or by administration of a radionuclide. IFIX also may be used with other gene-therapy regimes. In particular embodiments the IFIX is introduced into a tumor. The tumor may be in an animal, in particular, a human. The IFIX may be introduced by injection.

In some embodiments of the present invention, the inventor's discovery that IFIX is able to inhibit tumor cell proliferation will be used in combination with other therapeutic agents. The other therapies may be known at the time of this application, or may become apparent after the date of this application. IFIX may be used in combination with other therapeutic polypeptides, polynucleotides encoding other therapeutic polypeptides, chemotherapeutic agents, or radiotherapeutic agents. The IFIX composition may be introduced into a tumor, and the tumor may be contained in an animal, in particular, a human. The IFIX may be introduced by injection. In some embodiments, the other therapeutic agent induces apoptosis. In one preferred embodiment, the other agent capable of inducing apoptosis is TNFα. Other polypeptide inducers of apoptosis that may be used in combination with IFIX include, but are not limited to, p53, Bax, Bak, Bcl-x, Bad, Bim, Bok, Bik, Bid, Harakiri, Ad E1B, Bad and ICE-CED3 proteases. In other embodiments, a chemotherapeutic agent capable of inducing apoptosis is used in combination with IFIX. In one preferred embodiment, the chemotherapeutic agent capable of inducing apoptosis is taxol. In another embodiment, radiotherapy comprising ionizing radiation is the other apoptosis-inducing therapeutic agent. The type of ionizing radiation may be selected from the group comprising x-rays, γ-rays, and microwaves. The ionizing radiation may be delivered by external beam irradiation or by administration of a radionuclide.

The IFIX gene products and polynucleotides of the present invention may also be introduced using any suitable method. A “suitable method” of introduction is one that places a IFIX gene product in a position to reduce the proliferation of a tumor cell. For example, injection, oral, and inhalation methods may be employed, with the skilled artisan being able to determine an appropriate method of introduction for a given circumstance. In some preferred embodiments, injection will be used. This injection may be intravenous, intraperitoneal, intramuscular, subcutaneous, intratumoral, intrapleural, or of any other appropriate form. Systemic administration of IFIX is suitable, in some embodiments.

In certain other aspects of the present invention there are provided therapeutic kits comprising in a suitable container a pharmaceutical formulation of an IFIX gene product or a polynucleotide encoding an IFIX gene product. Such a kit may further comprise a pharmaceutical formulation of a therapeutic polypeptide, polynucleotide encoding a therapeutic polypeptide, and/or chemotherapeutic agent.

An IFIX as used herein is defined as a IFIX polypeptide, or the corresponding polynucleotide encoding same, that comprises anti-cell proliferation activity, anti-tumor activity, pro-apoptotic activity, and/or a combination thereof that is useful for the purposes described herein.

The term “IFIX” as used herein refers to an IFIX polynucleotide or polypeptide from any organism. In a specific embodiment, the IFIX comprises anti-tumor activity, anti-cell proliferation activity, pro-apoptotic activity and/or cell cycle arrest-inducing activity. For embodiments wherein IFIX employs tumor suppressive activity, the activity may relate to the tumor suppressor activity of IFN-γ, which upregulates expression of IFIX, as demonstrated herein. Consistent with the embodiment of IFIX comprising tumor suppressor activity, the present inventors demonstrate in at least Example 4 that IFIX suppresses cell growth, particularly in breast cancer cells.

The anti-tumor activity, anti-cell proliferation activity, pro-apoptotic activity and/or cell cycle-inducing activity may be useful for an organism other than the one from which the IFIX is derived. For example, the human IFIX is utilized herein in exemplary embodiments, although a murine IFIX may be used alternatively or in addition for human treatment. The IFIX from any organism may be altered at any amino acid. Furthermore, the human IFIX may be mutated at a particular residue(s) and found useful for therapy, and the mutant murine IFIX with its analogous residue(s) substitution may also be effective.

Of course, IFIX may be mutated for any number of reasons, and one of skill in the art is aware that there may be desirable mutations generated in the IFIX polypeptide or a nucleic acid encoding same that are for purposes other than removing phosphorylation sites and/or for effecting or retaining anti-tumor activity, anti-cell proliferation activity, and/or pro-apoptotic activity. For example, mutations may be made to render the IFIX polynucleotide and/or polypeptide more amenable for a therapeutic purpose. For example, modifications may be made that reduce antigenicity of the polypeptide, that remove regions of the polypeptide, that enhance nuclear localization of the polypeptide, that increase the half-life of the polypeptide, and so forth. At least one assay for determining effectiveness of a particular non-wildtype form of IFIX in comparison to wild type is described herein, and others are known in the art.

Specifically, the present invention is directed to methods and compositions regarding particular forms of IFIX that are associated with control of cell growth, survival or proliferation. In specific embodiments, the control of cell growth is useful in the treatment of cancer or restenosis. Specifically, the present invention teaches a skilled artisan that IFIX polypeptides are useful for anti-tumor applications.

In some embodiments, the invention relates to isolated polynucleotides having a region that comprises a sequence of SEQ ID NO:1, SEQ ID NO:2, SEQ ID NO:3 or SEQ ID NO:4, a complement of any of these sequences, or fragments thereof. In some more specific embodiments, the invention relates to such polynucleotides comprising a region having a sequence comprising at least 17, 20, 25, 30, 35, 40, 45, 50, 60, 70, 80, 90, 100, 125, 150, 200, or more contiguous nucleotides in common with at least one of SEQ ID NO: 1, SEQ ID NO:2, SEQ ID NO:3, or SEQ ID NO:4, or its complement. Of course, such polynucleotides may comprise a region having all nucleotides in common with at least one of SEQ ID NO: 1, SEQ ID NO:2, SEQ ID NO: 3, or SEQ ID NO:4.

In other aspects, the invention relates to polypeptides having sequences of SEQ ID NO:5, SEQ ID NO:6, SEQ ID NO:7, SEQ ID NO:8 or fragments thereof. The invention also relates to methods of producing such polypeptides using recombinant methods, for example, using the polynucleotides described above.

The invention also relates to antibodies against IFIX antigens, including those directed against an antigen having sequences of SEQ ID NO:5, SEQ ID NO:6, SEQ ID NO:7, or SEQ ID NO:8 or antigenic fragments thereof. The antibodies may be polyclonal or monoclonal and may be produced by methods known in the art.

One of skill in the art recognizes that mutations may be made in IFIX, and that some of these mutants will have the same activities as the exemplary embodiments provided herein. A skilled artisan is aware of publicly available databases that provide IFIX sequences, such as the National Center for Biotechnology Information's GenBank database or commercially available databases such as from Celera Genomics, Inc. (Rockville, Md.). For example, in specific embodiments an exemplary IFIX polynucleotide (followed by its GenBank Accession number) comprises IFIX-α1 (SEQ ID NO:1; AY185344); IFIX-α2 (SEQ ID NO:2; AY185345); IFIX-β1 (SEQ ID NO:3; AY185346); and/or IFIX-β2 (SEQ ID NO:4; AY185347). Exemplary IFIX polypeptides (followed by its GenBank Accession number) comprise IFIX-α1 (SEQ ID NO:5; AY185344); IFIX-α2 (SEQ ID NO:6; AY185345); IFIX-β1 (SEQ ID NO:7; AY185346); and/or IFIX-β2 (SEQ ID NO:8; AY 185347). Another exemplary IFIX polypeptide comprises IFIX γ1 (SEQ ID NO:9; XM086611).

Thus, the present invention provides guidance regarding IFIX, and, therefore, the present invention is directed to a novel improvement to the overall arts of cell growth control, including inhibition of cell proliferation and/or facilitation of cell death. In a specific embodiment, the inhibition of a cell proliferation comprises a delay in its rate of proliferation, a delay in its total cell numbers of proliferation, or both.

Therefore, an object of the present invention is directed to IFIX and, in some embodiments, to at least one modification in an IFIX, both of which the IFIXs comprise anti-cell proliferation capability, anti-tumor capability, pro-apoptotic capability, tumor suppressor activity, cell cycle arrest-inducing activity, or a combination thereof. In a specific aspect of the invention, the IFIX polypeptide is localized to the nucleus of a cell. More specifically, in some methods employing delivery of IFIX to a cell, the IFIX polypeptide is preferably delivered to the nucleus of the cell. Such delivery may be facilitated when the IFIX polypeptide comprises a nuclear localization signal, such as, for example, the exemplary SEQ ID NO:22 and SEQ ID NO:23.

A skilled artisan recognizes that any site in the IFIX polypeptide may be modified to generate such compositions as described, and furthermore that multiple sites may be modified. A skilled artisan is cognizant that a limited number of sites for modification exist in the IFIX polypeptide. In addition, a skilled artisan recognizes that there are only twenty standard amino acids from which to modify to, and guidance is provided herein directed to methods to generate those modifications. Furthermore, a skilled artisan in the teachings of the present invention knows how to test for anti-tumor, anti-cell growth, and/or pro apoptotic effects, and therefore assaying a particular modification would not subject one skilled in the art to undue experimentation.

Thus, based on the guidance provided herein, the present invention is directed to polypeptides of IFIX and/or polynucleotides encoding same that result in inhibition of proliferation of a cell or enhancement of cell death.

Thus, in accordance with the objects of the present invention, there is as a composition of matter an IFIX polypeptide. For example, the composition comprises the IFIX sequences provided herein or IFIX forms comprising at least one amino acid substitution. In other specific embodiments, the compositions are further defined as compositions in a pharmacologically acceptable excipient in which the IFIX polypeptide is dispersed. In additional specific embodiments, the compositions are confined in a suitable container in a kit.

In an additional object of the present invention, there is a method of preventing growth of a cell in an individual comprising the step of administering to the individual an IFIX polypeptide. In another specific embodiment, the administration of the polypeptide is by a liposome. In an additional specific embodiment, the polypeptide further comprises a protein transduction domain.

In another object of the present invention there is a method of preventing growth of a cell in an individual comprising the step of administering to the individual a nucleic acid encoding an IFIX polypeptide. In another specific embodiment, the administration of the nucleic acid is by a vector selected from the group consisting of a plasmid, a retroviral vector, an adenoviral vector, an adeno-associated viral vector, a liposome, and a combination thereof.

In an additional object of the present invention, there is a method of using an IFIX polypeptide composition wherein the IFIX polypeptide composition is dispersed in a pharmacologically acceptable excipient, and wherein the composition is administered to an animal having a proliferative cell disorder.

In another object of the present invention, there is a method of treating a proliferative cell disorder in an individual comprising the step of administering to the individual an IFIX polypeptide. In another specific embodiment, the proliferative cell disorder is cancer. In a further specific embodiment, the proliferative cell disorder is restenosis. In a further specific embodiment, the cancer is breast cancer, prostate cancer, or ovarian cancer.

In an additional object of the present invention, there is a method of treating a cell comprising contacting the cell with an IFIX polypeptide. In a specific embodiment, the cell is a human cell. In another specific embodiment, the cell is comprised in an animal. In a further specific embodiment, the animal is a human. In a further specific embodiment, the human has a proliferative cell disorder. In an additional specific embodiment, the proliferative cell disorder is cancer. In a further specific embodiment, the cancer is breast cancer, ovarian cancer, or prostate cancer. In another specific embodiment, the proliferative cell disorder is restenosis.

In another embodiment of the present invention, there are methods and compositions related to diagnosis of a disease utilizing a polynucleotide and/or polypeptide as described herein. For example, a sample from an individual with a disease or at risk for developing a disease is obtained, and the sample is assayed for an IFIX polynucleotide and/or polypeptide. In some embodiments, levels of the IFIX polynucleotide and/or polypeptide increase with a disease state, whereas in alternative embodiments levels of the IFIX polynucleotide and/or polypeptide decrease with a disease state. The sample obtained may be from blood, sputum, mucus, cheek scrapings, nipple aspirate, saliva, feces, urine, and any other entity from the body of the individual so long as it may be assayed for an IFIX polynucleotide and/or polypeptide. The IFIX polynucleotide and/or polypeptide and/or the levels thereof may be assayed for having at least one mutation, and the sample IFIX polynucleotide and/or polypeptide may be compared against wild-type controls. In a specific embodiment, the disease for which at least one IFIX polynucleotide and/or polypeptide is assayed is cancer.

A sample may be obtained from a human cancer, such as a solid tumor, and the sample is assayed by any suitable means in the art, such as, for example, PCR, northern, immunoassay, and so forth, and the level of an IFIX polynucleotide and/or polypeptide is determined. In a specific embodiment, a level of an IFIX polynucleotide and/or polypeptide is decreased in a sample, and this decrease is indicative of cancer. Thus, in some embodiments, an IFIX polynucleotide and/or polypeptide serves as a diagnostic and/or prognostic marker for cancer. In some embodiments, a kit comprising means to assay an IFIX polynucleotide and/or polypeptide is provided, particularly for the diagnosis and/or prognosis of cancer. The kit may comprise primers for polymerase chain reaction of an IFIX polynucleotide, antibodies for immunoassay of an IFIX polypeptide, or both. The kit may also comprise a means to obtain a sample from an individual, such as a syringe, tweezers, scalpel, and so forth. The kit may furthermore comprise an IFIX polynucleotide and/or polypeptide itself.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows structural comparison among HIN-200 proteins. The type a and type b 200-amino acid signature motifs of HIN-200 proteins are indicated by black and gray bars respectively. Different patterns of the C-terminus of IFIX isoforms indicate different amino acid sequences of their C-terminal S/T/P-rich domains. The black vertical bars in IFIX indicate the 9 amino acids absent in α2, β2, and γ2 isoforms.

FIGS. 2A and 2B show reduced expression of IFIX in breast cancer cells. FIG. 2A shows reduction of IFIX mRNA levels in breast cancers. The IFIX mRNA levels in normal breast (N) and breast cancer (T) tissues from five breast cancer patients were determined by RT-PCR. C, an IFIX cDNA clone used as the template in PCR. RT-PCR of GAPDH was used a control for the RNA quality. FIG. 2B shows reduction of IFIX mRNA levels in breast cancer cell lines. IFIX mRNA in 20 μg of total RNA isolated from indicated cell lines was determined using Northern blot analysis. GAPDH mRNA on the same blot was subsequently detected to serve as a RNA loading control.

FIGS. 3A through 3D show suppression of the growth and tumorigenicity of breast cancer cells by IFIX. FIG. 3A illustrates expression of exogenous IFIX in breast cancer cell lines. The Flag-tagged IFIX in lysates from Flag-tagged IFIX expressing clones (X-1 and X-2) and a control clone (C) were detected by western blot using an anti-Flag antibody. The actin protein levels serve as loading controls. FIG. 3B shows reduced growth rates in IFIX stable cell lines. The growth rate of clones expressing Flag-tagged IFIX (X-1 and X-2) and a control clone (C) was measured by MT assay. Each measurement was made in quadruplicate. FIG. 3C demonstrates suppression of in vitro transformation by IFIX. Parental (C) or IFIX stable cell lines (X-1 and X-2) derived from MDA-MB-468 or MCF-7 cells as indicated were seeded in soft agar and the colony number was scored at 3 weeks (MDA-MB-468), 5 weeks (468-X-1 and 468-X-2) or 4 weeks (MCF-7, MCF-X-1, and MCF-X-2) after seeding. Colony numbers of IFIX expressing clones are compared to that of their parental cells. FIG. 3D shows suppression of tumorigenicity by IFIX. MDA-MB-468 and 468-X-2 cells were implanted into the MFP of 6-week old female nude mice at two sites per mouse, 3 mice per group. The average tumor sizes at indicated time points are presented.

FIG. 4 demonstrates the anti-tumor effect of IFIX/SN2 liposome treatment in an orthotopic breast cancer xenograft model. Orthotopic breast tumors were established by inoculating MDAMB-468 cells into MFP of nude mice and the treatments began when tumors were about 0.5 cm in diameter. Tumors were treated twice weekly with SN2 mixed with either CMV-IFIX (X) or pCMV-Tag2B (V). The actual size of each tumor at the indicated time points during the treatment is presented. The average tumor size is indicated by horizontal bars. t-test: *p=0.1, ** p<0.0001, *** p<0.000005, and **** p<0.000002.

FIGS. 5A through 5C demonstrate up-regulation of p21^{CIP1} by IFIX. FIG. 5A shows increased p21^{CIP1} protein levels in MDAMB-468 and MCF-7 IFIX stable cells. Cell lysates isolated from 468-X-2, MCF-X-2, and the control cell lines (C) were analyzed by western blot using an anti-p21^{CIP1} antibody. Actin served as the loading control. FIG. 5B shows increased p21^{CIP1} mRNA levels in MDA-MB-468 and MCF-7 IFIX stable cells. Total RNA (20 μg) isolated from 468-X-2, MCF-X-2, and their control cell lines (C) were analyzed by northern blot using an IFIX or p21^{CIP1} probe as indicated. The 18S and 28S rRNA bands on the membrane stained by ethidium bromide serve as loading control. FIG. 5C shows inhibition of the kinase activity of CDK2 and p34^{CDC2} by IFIX. Cell lysates isolated from 468-X-2, MCF-X-2, and their control cell lines (C) were immunoprecipitated by CDK2 (or p34^{CDC2}) specific antibody followed by histone H1 (H1) kinase assay. Immunoprecipitation followed by western blot (IP/W) with CDK2 or p34^{CDC2} antibody served as the loading control.

FIGS. 6A through 6D show structures of IFIX isoforms. FIG. 6A shows schematics of the IFIX gene. Exons of the IFIX gene are shown as shaded boxes and the exon numbers are indicated, the size of exons and introns are not drawn in scale. Alternative splicing events that result in various IFIX isoforms are indicated. Arrow indicates a putative transcriptional start site determined by 5′ Rapid Amplification of cDNA Ends. AUG: the translation initiation codon; K: the highly charged, lysine-rich N-terminal domain; Δ27: the 27-bp absent in the α2, β2, and γ2 isoforms; shaded hexagons: stop codons of the γ, α, and β forms; PA: polyadenylation signal; MFHATVAT (SEQ ID NO: 15): an amino acid sequence shared among HIN-200 proteins; a: the type a 200-amino acid repeat; STP: serine/threonine/proline-rich region. FIG. 6B shows the 9 amino acids absent in isoforms α2, β2, and γ2. FIG. 6C shows the C-terminal amino acid sequences of isoforms α and β. Amino acids different between isoforms α and β are italicized. FIG. 6D provides the unique C-terminal amino acid sequence of the γ isoforms. Amino acids of γ isoforms different from isoforms α and β are italicized. The MFHATVAT signature motif of HIN-200 proteins in α and β isoforms is underlined.

FIG. 7 provides amino acid sequence comparison among human HIN-200 proteins. Amino acids identical in at least two sequences are highlighted. Dashes indicate gaps introduced in the sequence to obtain the best alignment.

FIGS. 8A and 8B show tissue distribution and IFN induction of IFIX expression. FIG. 8A shows induction of IFIX expression by IFN. The IFIX mRNA in indicated cell lines without treatment (c) or treated with 100 u/ml of IFN-α (α) or IFN-γ (γ) was detected by northern blot analysis. The 18S and 28S rRNAs serve as loading controls. FIG. 8B demonstrates that IFIX expresses mainly in the secondary lymphoid organs. The Multiple Tissue Northern blot (Clontech; Palo Alto, Calif.) was hybridized with an IFIXα1 cDNA probe. The IFIX mRNA (IFIX) and an unknown band (?) are indicated. The actin mRNA served as the loading control. Sp: spleen; LN: lymph node; PBL: peripheral blood leukocyte; Thy: thymus; BM: bone marrow; FL: fetal liver.

FIG. 9 demonstrates reduced expression of IFIX in human cancers. The Matched Tumor/Normal Expression Array was first hybridized with an IFIX probe and then with an ubiquitin probe. The hybridization results of breast (n=50) and ovarian (n=14) tissues are shown. The signal of IFIX was normalized with that of ubiquitin and samples in which IFIX is down-regulated are indicated by arrows. N: normal; T: tumor.

FIG. 10 illustrates that IFIX is downregulated in breast cancer cells. IFIX protein expression was monitored by western blot with IFIX-antibody. The normal breast cell lines, e.g., MCF-10A and MCF-12A, have detectable IFIX expression. Breast cancer cell lines, MDA-MB-468 (468), ZR-75-1, T47 D, HBC 100, MDA-MB-231 (231), MCF-7, and MDA-MB-453 (453) have undetectable IFIX expression. MDA-MB-435 has low level of IFIX expression. α-tubulin serves as the loading control.

FIG. 11 shows that IFIX is inducible in breast cancer cell lines. IFIX mRNA expression was monitored in SKBR3, MDA-MB-231, and MCF-7 with or without IFN-α (100 u/ml) for 20 h (or IFN-γ (100 u/ml) in MCF-7) by RT-PCR using IFIX-specific primers. IFIX protein is induced upon IFN-γ treatment. MCF-7 cells were treated with or without IFN-γ (1000 u/ml) for 72 h, followed by western blot using anti-IFIX specific antibody generated by the inventors. IFIX can also be induced after IFN-α (1000 u/ml) treatment for 72 h.

FIG. 12 demonstrates IFIX promoter and deletion mutants. IFIX promoter was cloned using two overlapping primer sets (S1/AS1 and S2/AS2). Five putative transcriptional start sites (indicated by diamonds) were determined using the 5′-RACE kit (Clontech; Palo Alto, Calif.) to analyze the cDNA obtained from Daudi cells. The solid diamond indicates the first potential trancription initiation site (+1). Computer search identified several putative transcriptional factor binding sites that include several housekeeping transcriptional factor binding sites, e.g., SP1(2) and TATA (3) regulatory factor binding sites, e.g., IFN-stimulated responsive elements (ISRE) (1), STAT1 (5), NFκB (4) and estrogen receptor (ER) (6). The unique restriction sites for generating deletion mutants are indicated.

FIG. 13 illustrates effects on tumor volume in a prostate cancer mouse model treated with CMV-IFIX and empty vector control.

FIG. 14 shows reduced expression of IFIX in human breast tumors. The commercially available human cDNAs derived from 12 normal breast tissues (Normal) and 12 breast carcinoma tissues (Tumor) (Origene Technologies, Inc.) were analyzed for IFIX expression by PCR using primers specific to IFIXα. The IFIXα1 cDNA was used as a control (C). Molecular weight markers (M) are indicated. The IFIXα and β-actin specific bands are indicated. NS: non-specific PCR products. Samples positive for IFIX? expression are indicated by solid triangles.

FIG. 15 demonstrates the presence of IFIX isoforms in the IFIX-expressing cell lines. RT-PCR was performed using primers specific for IFIXα, β, (top panel) or γ (middle panel), and the “form 2” (indicated by an arrowhead, bottom panel) in Daudi, MCF-10A (10A), MCF-12A (12A), MDA-MB-231 (231), and MDA-MB-435 (435). The IFIXα1, α2, β1, and γ1 cDNAs were used as controls.

FIG. 16 shows suppression of the growth by IFN correlates with IFIX induction. IFN-γ induces IFIX expression in breast cancer cells. Top panel: MCF-7 and MDA-MB-468 cells were treated with (open bars) or without (solid bars) IFN-γ (1000 U/ml) in DMEM/F12 media containing 0.25% fetal calf serum for 48 hours. The growth of the cells was measured by MTT assay. The experiment was run in triplicate and represented as the mean±SD. The asterisks represent the statistically significant differences due to the IFN-γ treatment. * p<0.0005, ** p<0.036. Bottom panel: Total RNAs isolated from MCF-7 and MDA-MB-468 cells treated with or without IFN-γ (1000 U/ml) under the same condition as described above were analyzed for IFIX expression by RT-PCR. GAPDH was used as an internal control. The IFIXα1 cDNA was used as a specificity control (C). Molecular weight markers (M) are indicated.

FIG. 17 shows that IFIX expression affects cell cycle distribution. The IFIX-expressing cells (X-2) and the empty vector control cells (V) derived from MCF-7 and MDA-MB-468 cells were subjected to flow cytometry analysis. The percentage of each cell line in G1-, S-, and G2/M-phases was calculated. This result was obtained from two independent experiments.

FIGS. 18A and 18B demonstrate nuclear localization of IFIX. In FIG. 18A, the stably transfected IFIXα1 protein is localized in the nucleus. Cytoplasmic (C), nuclear (N), or whole cell extract (WCE) were isolated from MCF-X-1, MCF-X-2, or the MCF-7 empty vector control cells (V) were analyzed for IFIXα1 expression by western blot using an anti-Flag antibody. The same blot was used to verify the quality of the extracts using the antibodies against the nuclear protein, PARP, and the cytoplasmic protein, α-Tubulin. In FIG. 18B, the transiently transfected IFIXα1 protein is localized in the nucleus. MCF-7 cells were transfected with the plasmid encoding EGFP-tagged IFIXα1, β1, or γ1 protein. The EGFP-expression vector serves as a control. Phase contrast, nuclear staining (DAPI), green fluorescence (FITC), and Texas Red for p21CIP1 staining (p21) of each transfection are shown. Forty-eight hours after transfection, the percentage of p21CIP1-positive in EGFP-positive cells was counted for each transfection: EGFP (0.95%, 1/105), IFIXα1 (64%, 68/107), IFIXβ1 (52%, 55/106), and IFIXγ1 (2%, 2/100). Cells were examined at 60× magnification.

FIGS. 19A-19C show that IFIXα1 activates p53 by downregulating HDM2. In FIG. 19A, IFIXα1 increases HDM2 mRNA levels. Total RNA (10 μg) isolated from MCF-7 parental (P), vector (V) control, and IFIXα1 stable cell lines (X-1 and X-2) were analyzed by northern blot using an HDM2, p53, or IFIXα1 probe as indicated. In FIG. 19B, IFIXα1 increases p53 protein levels. Total cell lysates were analyzed by western blot using antibodies against HDM2, p53, IFIXα1, and α-tubulin (as a loading control). In FIG. 19C, IFIXα1 enhances p53 DNA binding activity. Nuclear extracts (NE) (7.5 μg) isolated from each cell were incubated with ³²P-labeled oligonucleotide containing p53-binding sites prior to electrophoretic mobility shift assay according to manufacturer's instruction (p53 Nushift kit; Geneka Biotechnology, Inc.; Montreal, Quebec).

FIGS. 20A-20D show that IFIXα1 promotes HDM2 protein degradation. In FIG. 20A, the HDM2 protein levels are not p53 dose-dependent in the presence of IFIXα1. H1299 cells were transfected with increased amount of IFIXα1 (0.5, 1.0, 1.8 μg) and p53 (0.1 μg). Twenty-four h post-transfection, cell lysates were isolated for western blot analysis using antibodies against HDM2, p53, IFIXα1, p21^{CIP1}, and α-tubulin (as a loading control). In FIG. 20B, IFIXα1 reduces HDM2 protein levels in the absence of p53. H1299 cells transfected with with pcDNA3 (Vector) (1.5 μg), HDM2 (0.8 μg)+Vector (0.7 μg), or HDM2 (0.8 μg)+IFIXα1 (0.7 μg). Forty h post-transfection, cell lysate was isolated followed by western blot using anti-HDM2, anti-IFIX, or anti-a-tubulin antibody. In FIG. 20C, IFIXα1 expression reduces the HDM2 levels. 293T cells were transfected with 2 μg of either EGFP vector (V) or EGFP-IFIXα1 (α1) followed by western blot using anti-HDM2, anti-IFIX, or anti-a-tubulin antibody. In FIG. 20D, IFIXα1 reduces the half-life of HDM2 protein. H1299 cells were co-transfected with HDM2 (0.7 μg) and 1.3 μg of either pcDNA3 (V) or IFIXα1. Twenty-four h post-transfection, cells were treated with cyclohexamide (CHX) (100 μg/ml). Cell lysates were isolated at 0, 15, and 30 min after CHX treatment for western blot analysis using antibodies against HDM2, IFIXα1, and α-tubulin (as a loading control).

FIGS. 21A through 21C demonstrate that IFIXα1 interacts with HDM2. In FIG. 2A, HDM2 interacts with EGFP-IFIXα1 and β1. 293T cells were transfected with 2.5 μg CMV-HDM2 and 2.5 μg CMV-vecter (Vector), or 2.5 μg EGFP-IFIXα1 (α1), EGFP-IFIXβ1 (β1). Forty-eight h post-transfection, cell extracts (500 μg) were immunoprecipitated (IP) with a monoclonal anti-HDM2 antibody (Santa Cruz), and western blot (WB) was performed using a polyclonal anti-GFP (Abcam) or anti-MDM2 (Santa Cruz) antibody. In FIG. 21B, there is a reciprocal experiment that used anti-GFP antibody for IP and WB with anti-IFIX or anti-HDM2 antibodies. In FIG. 21C, HDM2 interacts with Flag-tagged IFIXα1. 293T cells were transfected with 5 μg CMV-HDM2 and 5 μg Flag-vector (V) or Flag-IFIXα1 (α1). Forty-eight h post-transfection, cell extracts (500 μg) were immunoprecipitated using anti-Flag (M5, Sigma), anti-IFIX, or anti-HDM2 antibodies and WB using anti-IFIX, anti-Flag, and anti-IFIX antibody, respectively.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

Cancer is a genetic disease. Many genetic alterations are associated with, and some are the causes for, cancer. For instance, mutation or downregulation of tumor suppressor genes often plays an important role during tumorigenesis. Better understanding of these mutations and altered regulation leads to better cancer treatment.

The present invention relates to a novel member of human HIN-200 (Hematopoietic Interferon (IFN)—inducible Nuclear proteins with 200 amino-acid repeat) protein, named IFIX (stands for IFN-Inducible protein X). Although the sequence of the 200 amino acid repeat is diverse among HIN-200 proteins, the 200 amino acid repeat sequence of IFIXα1, α2, β1, and β2 is provided in SEQ ID NO:21, the sequence of which is as follows: MFHATVATQTQFFHVKVLNINLKRKFIKKRIIIISNYSKRNSLLEVNEASSVSEAGPDQTFEVP KDIIRRAKKIPKINILHKQTSGYIVYGLFMLHTKIVNRKTTIYEIQDKTGSMAVVGKGECHNIP CEKGDKLRLFCFRLRKRENMSKLMSEMHSFIQIQKNTNQRSHDSRSMALPQEQSQHPKPSEA STTLPESHLK. The underlined amino acid sequence is found in the 200 amino acid repeat of all known HIN-200 proteins. In some embodiments, the putative nuclear localization signal is ¹³⁶PQKRKK¹⁴¹ (SEQ ID NO:22) of the IFIXα1 protein, however in some embodiments a slightly larger nuclear localization sequence ¹³⁴LGPQKRKK¹⁴¹ (SEQ ID NO: 23) is utilized.

IFIX possesses a highly charged N-terminal domain typical to HIN-200 proteins and a type a 200-amino acid signature motif unique to this protein family. IFIX expression is readily inducible upon either type I (e.g., IFN-α) or type II (IFN-γ) IFN treatment in a variety of cell lines tested, including hematopoietic and breast cancer cell lines. Like most HIN-200 proteins, IFIX possesses a putative nuclear translocation signal (SEQ ID NO:22) and is a nuclear protein. Tissue distribution study indicates that IFIX expression is found mainly in the secondary lymphoid organs, such as spleen and lymph node, but not in the primary lymphoid organs, such as thymus and bone marrow. This observation strongly suggests that IFIX is involved in the immune response. Interestingly, IFIX expression is downregulated in tumors of the matched tumor/normal pairs from patients with cancer of ovary (11/14, 79%, breast (27/50-54%), prostate (2/4, 50%), lung (10/21, 48%), rectum (7/18, 39%), colon 11/34, 32%), kidney (7/20, 35%), thyroid (2/6,33%), uterus (12/42, 29%), and stomach (9/27,33%).

In specific embodiments of the present invention, there are multiple isoforms of IFIX. For example, in specific embodiments an exemplary IFIX polynucleotide comprises IFIX-α1 (SEQ ID NO:1); IFIX-α2 (SEQ ID NO:2); IFIX-β1 (SEQ ID NO:3); and/or IFIX-β2 (SEQ ID NO:4). Exemplary IFIX polypeptides comprise IFIX-α1 (SEQ ID NO:5); IFIX-α2 (SEQ ID NO:6); IFIX-β1 (SEQ ID NO:7); and/or IFIX-β2 (SEQ ID NO:8). Another exemplary IFIX polypeptide comprises IFIX γ1 (SEQ ID NO:9).

The present invention regards IFIX polypeptides and the nucleic acids that encode them, as well as methods regarding the use of IFIX.

In some embodiments, the IFIXs inhibit cell proliferation of a cancer cell or of a cell other than a cancer cell and is useful for the treatment of restenosis or to inhibit angiogenesis. One skilled in the art following the teachings of this specification can generate at least one of the wildtype IFIX forms and/or exemplary mutants thereof.

A skilled artisan recognizes that mutants of IFIX may be generated by a variety of means. In a specific embodiment, a nucleic acid sequence as set forth herein, for example, SEQ ID NO: 1 through SEQ ID NO:4, is mutated at the codon(s) that encodes a particular amino acid desired to be altered. Table 1 presents codons for all standard amino acids, and a skilled artisan would be well aware how to manipulate a starting nucleic acid to generate a desired mutation using standard site-directed mutagenesis techniques, for example.

In an embodiment of the present invention, the IFIX wild type gene product may be phosphorylated under native conditions, and in some embodiments a phosphorylation site(s) is mutated. For example, a serine or threonine amino acid residue may be changed by altering the nucleic acid codon that encodes it, such as by site-directed mutagenesis. Alternatively, the amino acid(s) for phosphorylation may be blocked with at least one compound that prevents phosphorylation, for example with blocking agents such as carbodiamide or by acetylation of the residue with acetylchloride in trifluoroacetic acid. A skilled artisan recognizes that the substitution at at least one phosphorylation site may prevent phosphorylation of the IFIX polypeptide under conditions that would result in phosphorylation of an unsubstituted IFIX polypeptide, and furthermore would know methods standard in the art to determine these conditions.

In other embodiments of the present invention, there are methods of preventing growth of a cell in an individual comprising administering to the individual an IFIX polypeptide. In specific embodiments, the polypeptide is administered in a liposome and/or the polypeptide further comprises a protein transduction domain (Schwarze et al., 1999). In alternative embodiments, IFIX is administered as a polynucleotide, wherein the polynucleotide comprises the alteration that effects modification at the amino acid level, such as is generated by site-directed mutagenesis. The modified IFIX polynucleotide is administered in a vector such as a plasmid, retroviral vector, adenoviral vector, adeno-associated viral vector, liposome, or a combination thereof.

There are also embodiments of the present invention wherein there are methods of treating a cell comprising contacting the cell with an IFIX polypeptide. In specific embodiments, the cell is a human cell, the cell is comprised in an animal, and/or the animal is human.

It is contemplated herein that the compositions of the present invention preferably have an activity similar from a native IFIX polypeptide in the cell, and for an IFIX mutant that may be approximately the same or more potent against a cancer cell than native IFIX. That is, the scope of the present invention, in some embodiments, is directed to a change in the native IFIX polypeptide for use in a manner similar to the wildtype IFIX polypeptide. In an alternative embodiment, the IFIX forms (e.g. different from the wild type sequence) comprise an activity different from the native IFIX polypeptide.

I. Definitions and Techniques Affecting IFIX Proteins, Polypeptides, Peptides, and the Nucleic Acids Encoding Them

A. IFIX Proteins, Polypeptides, Peptides, and the Nucleic Acids Encoding them

As used herein, the terms “IFIX gene product” and “IFIX” refer to proteins having amino acid sequences that may or may not be identical to the native IFIX but that are biologically active in that they are capable of performing similar activities to native IFIX. For example, they are preferably capable of pro-apoptotic activity, anti-cell proliferative activity, anti-tumor activity and/or cross-reactive antibody activity with anti-IFIX antibody raised against IFIX. The term “IFIX gene product” includes analogs of IFIX molecules that exhibit at least some biological activity in common with native IFIX. Furthermore, those skilled in the art of mutagenesis will appreciate that other analogs, as yet undisclosed or undiscovered, may be used to construct IFIX analogs.

The term “mutant form of IFIX” refers to any DNA sequence that is substantially identical to a DNA sequence encoding a IFIX gene product as defined above. The term also refers to RNA or antisense sequences compatible with such DNA sequences. A “IFIX gene” may also comprise any combination of associated control sequences.

In some embodiments of the present invention, the IFIX protein, polypeptide, peptide, and/or polynucleotide(s) encoding them are fragments and/or derivatives of a wild-type endogenous form, so long as they are active. In a specific embodiment, the fragments and/or derivatives have the identical or similar activity as the wild-type endogenous form. In a further specific embodiment, this activity is a tumor suppressor activity, an anti-cancer activity, a cancer therapeutic activity, a pro-apoptosis activity, an anti-cell proliferative activity, a combination thereof, and so forth.

The term “substantially identical”, when used to define either an IFIX amino acid sequence or IFIX nucleic acid sequence, means that a particular subject sequence, for example, a mutant sequence, varies from the sequence of natural IFIX by, for example, one or more substitutions, deletions, additions, or a combination thereof, the net effect of which is to retain at least some biological activity of the IFIX protein. Alternatively, DNA analog sequences are “substantially identical” to specific DNA sequences disclosed herein if: (a) the DNA analog sequence is derived from coding regions of the natural IFIX gene; or (b) the DNA analog sequence is capable of hybridization of DNA sequences of (a) under moderately stringent conditions and which encode biologically active IFIX; or (c) DNA sequences that are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequence.

A skilled artisan recognizes that an IFIX polypeptide may have about 50%, 55%, 60%, 65%, 70%, 75%, 80%, 85%, 90%, 95%, 96%, 97%, 98%, or 99% homology to at least one of a sequence of SEQ ID NO:5, SEQ ID NO:6, SEQ ID NO:7, or SEQ ID NO:8. A skilled artisan also recognizes that an IFIX polypeptide may have about 50%, 55%, 60%, 65%, 70%, 75%, 80%, 85%, 90%, 95%, 96%, 97%, 98%, or 99% sequence identity to at least one of a sequence of SEQ ID NO:5, SEQ ID NO:6, SEQ ID NO:7, or SEQ ID NO:8.

Furthermore, one of skill in the art recognizes that an IFIX polynucleotide may have about 50%, 55%, 60%, 65%, 70%, 75%, 80%, 85%, 90%, 95%, 96%, 97%, 98%, or 99% homology to at least one of a sequence of SEQ ID NO:5, SEQ ID NO:6, SEQ ID NO:7, or SEQ ID NO:8. A skilled artisan recognizes that an IFIX polynucleotide may have about 50%, 55%, 60%, 65%, 70%, 75%, 80%, 85%, 90%, 95%, 96%, 97%, 98%, or 99% identity to at least one of a sequence of SEQ ID NO:5, SEQ ID NO:6, SEQ ID NO:7, or SEQ ID NO:8.

B. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps.

C. Nucleic Acid Sequences

In certain embodiments, the invention concerns the use of IFIX nucleic acids, genes and gene products, such as the IFIX that includes a sequence that is different from that of the known IFIX gene, or the corresponding protein. The term “a sequence essentially as IFIX” means that the sequence substantially corresponds to a portion of the IFIX gene and has relatively few bases or amino acids (whether DNA or protein) that are not identical to those of IFIX (or a biologically functional equivalent thereof, when referring to proteins). The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of IFIX will be sequences that are “essentially the same”.

IFIX nucleic acids that have functionally equivalent codons are covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glutamic Acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCU | | | |
| Glutamine | Gln | Q | CAA | CAG | | | | |
| Arginine | Arg | R | AGA | AGG | CGA | CGC | GGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have at least about 50%, usually at least about 60%, more usually about 70%, most usually about 80%, preferably at least about 90% and most preferably about 95% of nucleotides that are identical to one of the IFIX nucleotides. Sequences that are essentially the same as those set forth in an IFIX gene or polynucleotide may also be functionally defined as sequences that are capable of hybridizing to a nucleic acid segment containing the complement of an IFIX polynucleotide under standard conditions.

The DNA segments of the present invention include those encoding functional and/or immunologically equivalent Chlamydia psittaci proteins and peptides, as described above. Such sequences may arise as a consequence of codon redundancy and amino acid functional equivalency that are known to occur naturally within nucleic acid sequences and the proteins thus encoded. Alternatively, functionally and/or immunogenically equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which may, for example, include various non-coding sequences flanking either of the 5′ or 3′ portions of the coding region or may include various internal sequences, i.e., introns, which are known to occur within genes.

The present invention also encompasses the use of DNA segments that are complementary, or essentially complementary, to the sequences set forth in the specification. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment in question under relatively stringent conditions such as those described herein.

D. Biologically Functional Equivalents

As mentioned above, modification and changes may be made in the structure of IFIX and still obtain a molecule having like or otherwise desirable characteristics. For example, certain amino acids may be substituted for other amino acids in a protein structure without appreciable loss of interactive binding capacity with targets. Since, in many embodiments, it is the interactive capacity and nature of a protein that defines that protein's biological functional activity, certain amino acid sequence substitutions can be made in a protein sequence (or, of course, its underlying DNA coding sequence) and nevertheless obtain a protein with like or even countervailing properties (e.g., antagonistic vs. agonistic). It is thus contemplated by the inventors that various changes may be made in the sequence of the IFIX proteins or peptides (or underlying DNA) without appreciable loss of their desired biological utility or activity.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged.

Amino acid substitutions, such as those that might be employed in modifying IFIX, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ^{±}2 is preferred, those that are within ^{±}1 are particularly preferred, and those within ^{±}0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those that are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

E. Oligonucleotide Sequences

Naturally, the present invention also encompasses DNA segments that are complementary, or essentially complementary, to the sequences of an IFIX polynucleotide. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of an IFIX polynucleotide under relatively stringent conditions such as those described herein. Such sequences may encode the entire IFIX polypeptide or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides will find use, for example, as probes in Southern and Northern blots and as primers in amplification reactions, or for vaccines.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

III. Nucleic Acid-Based Expression Systems

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

C. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

D. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

E. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

F. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

F. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

G. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

H. Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

I. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REx™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

IV. Nucleic Acid Delivery

The general approach to the aspects of the present invention concerning compositions and/or therapeutics is to provide a cell with a gene construct encoding a specific and/or desired protein, polypeptide and peptide, thereby permitting the desired activity of the proteins to take effect. While it is conceivable that the gene construct and/or protein may be delivered directly, a preferred embodiment involves providing a nucleic acid encoding a specific and desired protein, polypeptide and peptide to the cell. Following this provision, the proteinaceous composition is synthesized by the transcriptional and translational machinery of the cell, as well as any that may be provided by the expression construct. In providing antisense, ribozymes and other inhibitors, the preferred mode is also to provide a nucleic acid encoding the construct to the cell.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments and “episomes” encode sequences sufficient to permit maintenance and replication independent of and in synchronization with the host cell cycle. How the expression construct is delivered to a cell and/or where in the cell the nucleic acid remains is dependent on the type of expression construct employed.

A. DNA Delivery Using Viral Vectors

The ability of certain viruses to infect cells and enter cells via receptor-mediated endocytosis, and to integrate into host cell genome and/or express viral genes stably and/or efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and/or in the range of cells they infect, these viruses have been demonstrated to successfully effect gene expression. However, adenoviruses do not integrate their genetic material into the host genome and/or therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles and endotoxins and other pyrogens such that it will not cause any untoward reactions in the cell, animal and/or individual receiving the vector construct. A preferred means of purifying the vector involves the use of buoyant density gradients, such as cesium chloride gradient centrifugation.

B. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and/or (b) to ultimately express a tissue and/or cell-specific construct that has been cloned therein.

The expression vector comprises a genetically engineered form of adenovirus. Knowledge of the genetic organization and adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and/or no genome rearrangement has been detected after extensive amplification.

Adenovirus is particularly suitable for use as a gene transfer vector because of its mid-sized genome, ease of manipulation, high titer, wide target-cell range and/or high infectivity. Both ends of the viral genome contain 100-200 base pair inverted repeats (ITRs), which are cis elements necessary for viral DNA replication and/or packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and/or E2B) results in the synthesis of the proteins for viral DNA replication. These proteins are involved in DNA replication, late gene expression and/or host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and/or all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and/or examine its genomic structure.

Generation and/or propagation of the current adenovirus vectors, which are replication deficient, depend on a unique helper cell line, designated 293, which was transformed from human embryonic kidney cells by Ad5 DNA fragments and constitutively expresses E1 proteins (E1A and/or E1B; Graham et al., 1977). Since the E3 region is dispensable from the adenovirus genome (Jones and Shenk, 1978), the current adenovirus vectors, with the help of 293 cells, carry foreign DNA in either the E1, the D3 and both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from human cells such as human embryonic kidney cells, muscle cells, hematopoietic cells and other human embryonic mesenchymal and epithelial cells. Alternatively, the helper cells may be derived from the cells of other mammalian species that are permissive for human adenovirus. Such cells include, e.g., Vero cells and other monkey embryonic mesenchymal and/or epithelial cells. As stated above, the preferred helper cell line is 293.

Racher et al. (1995) disclosed improved methods for culturing 293 cells and/or propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and/or manipulation is known to those of skill in the art, and/or exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and/or therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991 a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and/or stereotactic inoculation into the brain (Le Gal La Salle et al., 1993). Recombinant adenovirus and adeno-associated virus (see below) can both infect and transduce non-dividing human primary cells.

C. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and/or U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and/or in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus and a member of the herpes virus family) to undergo a productive infection in cultured cells (Muzyczka, 1992). In the absence of coinfection with helper virus, the wild type AAV genome can integrate through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome may be “rescued” from the chromosome and from a recombinant plasmid, and/or a normal productive infection is established (Samulski et al., 1989; McLaughlin et al., 1988; Kotin et al., 1990; Muzyczka, 1992).

Typically, recombinant AAV (rAAV) virus can be made by cotransfecting a plasmid containing the gene of interest flanked by the two AAV terminal repeats (McLaughlin et al., 1988; Samulski et al., 1989; each incorporated herein by reference) and/or an expression plasmid containing the wild type AAV coding sequences without the terminal repeats, for example pIM45 (McCarty et al., 1991; incorporated herein by reference). The cells can also be infected and transfected with adenovirus and plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and cell lines containing the AAV coding regions and some and all of the adenovirus helper genes could be used (Yang et al., 1994; Clark et al., 1995). Cell lines carrying the rAAV DNA as an integrated provirus can also be used (Flotte et al., 1995).

D. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA can then stably integrate into cellular chromosomes as a provirus and/or directs synthesis of viral proteins. The integration can result in the retention of the viral gene sequences in the recipient cell and/or its descendants. The retroviral genome contains three genes, gag, pol, and/or env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest may be inserted into the viral genome in the place of certain viral sequences to produce a virus that is replication-defective. In order to produce virions, a packaging cell line containing the gag, pol, and env genes but without the LTR and packaging components may be constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses can then be collected, optionally concentrated, and used for gene transfer. Retroviral vectors are able to infect a broad variety of cell types. However, integration and/or stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

E. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and/or herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and/or can be grown to high titers. Thus, delivery is less of a problem, both in terms of volumes needed to attain sufficient MOI and in a lessened need for repeat dosings.

F. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and/or against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

G. Other Methods of DNA Delivery

In various embodiments of the invention, DNA is delivered to a cell as an expression construct. In order to effect expression of a gene construct, the expression construct must be delivered into a cell. As described herein, the preferred mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA and/or plasmids. Transfer of the construct may be performed by any of the methods mentioned which physically and/or chemically permeabilize the cell membrane. Some of these techniques may be successfully adapted for in vivo and/or ex vivo use, as discussed below.

1. Liposome-Mediated Transfection

In a further embodiment of the invention, the expression construct may be entrapped in a liposome. Liposomes are vesicular structures characterized by a phospholipid bilayer membrane and/or an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and/or entrap water and/or dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and/or expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and/or promote cell entry of liposome-encapsulated DNA (Kaneda et al., 1989). In other embodiments, the liposome may be complexed and/or employed in conjunction with nuclear non-histone chromosomal proteins (HMG-1) (Kato et al., 1991). In yet further embodiments, the liposome may be complexed and/or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

The inventors contemplate that neu-suppressing gene products can be introduced into cells using liposome-mediated gene transfer. It is proposed that such constructs can be coupled with liposomes and directly introduced via a catheter, as described by Nabel et al. (1990). By employing these methods, the neu-suppressing gene products can be expressed efficiently at a specific site in vivo, not just the liver and spleen cells which are accessible via intravenous injection. Therefore, this invention also encompasses compositions of DNA constructs encoding a neu-suppressing gene product formulated as a DNA/liposome complex and methods of using such constructs.

As described in U.S. Pat. No. 5,641,484, liposomes are particularly well suited for the treatment of HER2/neu-mediated cancer

2. Preparation of Liposomes

Catatonic liposomes that are efficient transfection reagents for IFIX for animal cells can be prepared using the method of Gao et al. (1991). Gao et al. describes a novel catatonic cholesterol derivative that can be synthesized in a single step. Liposomes made of this lipid are reportedly more efficient in transfection and less toxic to treated cells than those made with the reagent Lipofectin. These lipids are a mixture of DC-Chol (“3β(N-(N′N′-dimethylaminoethane)-carbamoyl cholesterol”) and DOPE (“dioleoylphosphatidylethanolamine”). The steps in producing these liposomes are as follows.

DC-Chol is synthesized by a simple reaction from cholesteryl chloroformate and N,N-Dimethylethylenediamine. A solution of cholesteryl chloroformate (2.25 g, 5 mmol in 5 ml dry chloroform) is added dropwise to a solution of excess N,N-Dimethylethylenediamine (2 ml, 18.2 mmol in 3 ml dry chloroform) at 0° C. Following removal of the solvent by evaporation, the residue is purified by recrystallization in absolute ethanol at 4° C. and dried in vacuo. The yield is a white powder of DC-Chol.

Cationic liposomes are prepared by mixing 1.2 μmol of DC-Chol and 8.0 μmol of DOPE in chloroform. This mixture is then dried, vacuum desiccated, and resuspended in 1 ml sterol 20 mM Hepes buffer (pH 7.8) in a tube. After 24 hours of hydration at 4° C., the dispersion is sonicated for 5-10 minutes in a sonicator form liposomes with an average diameter of 150-200 nm.

To prepare a liposome/DNA complex, the inventors use the following steps. The DNA to be transfected is placed in DMEM/F12 medium in a ratio of 15 μg DNA to 50 μl DMEM/F12. DMEM/F12 is then used to dilute the DC-Chol/DOPE liposome mixture to a ratio of 50 μl DMEZM/F12 to 100 μl liposome. The DNA dilution and the liposome dilution are then gently mixed, and incubated at 37° C. for 10 minutes. Following incubation, the DNA/liposome complex is ready for injection.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3.beta[N-(N′N′-dimethylaminoethane)-carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al., 1987, Nabel et al., 1990, and Gao et al., 1991. In a specific embodiment, the liposomes comprise DC-Chol. More particularly, one may utilize the liposomes comprising DC-Chol and DOPE that have been prepared following the teaching of Gao et al. (1991) in the manner described in the Preferred Embodiments Section. The inventors also anticipate utility for liposomes comprised of DOTMA, such as those which are available commercially under the trademark Lipofectin™, from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allow liposome-mediated transfer of DNA complex, for example, the liver and the spleen. In order to allow transfection of DNA into cells which are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding a nucleic acid encoding a form of IFIX.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is anticipated to have utility, it is anticipated that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

In a specific embodiment, one employs the smallest region needed to enhance retention of IFIX in the nucleus of a cell so that one is not introducing unnecessary DNA into cells which receive a IFIX gene construct. Techniques well known to those of skill in the art, such as the use of restriction enzymes, will allow for the generation of small regions of IFIX. The ability of these regions to inhibit neu can easily be determined by the assays reported in the Examples.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

3. In Vivo Treatment of Cancer Via Liposomes with IFIXs

Based on the teachings provided herein, a skilled artisan recognizes that any cell may be treated with at least one IFIX, and in particular embodiments, any cancer cell may be treated with such. For example, in some embodiments the nature of the treated cell is irrespective of being HER2/neu-positive or HER2/neu-negative. However, in one specific embodiment it is HER2/neu-positive.

U.S. Pat. No. 5,641,484, incorporated in its entirety by reference herein, teaches that liposome-mediated direct gene transfer techniques can be employed to obtain suppression of HER2/neu-overexpressing human cancer cells in living host. The protocol for described therein was as follows. Female nude mice (5-6 weeks old) were given intraperitoneal injections of SK-OV-3 cells (2×10⁶/100 μl). SK-OV-3 cells are human ovarian cancer cells that have been shown to grow within the peritoneal cavity of nude mice. After five days, the mice were given intraperitoneal injections of various compounds. Some mice were injected with the therapeutic DNA alone, some were injected with liposome/therapeutic DNA complex prepared in the manner described above, and some were injected with liposome/mutant therapeutic DNA complex. 200 μl of a given compound was injected into a given mouse. After the initial injections, injections were repeated every seven days throughout the life of the mouse.

The results described therein indicate that liposome-mediated gene transfer can inhibit HER2/neu-overexpressing human ovarian cancer cell growth. Therefore, it is predictable that liposome-mediated IFIX gene therapy may serve as a powerful therapeutic agent for HER-2 neu-overexpressing human ovarian cancers by direct targeting of IFIX at the HER-2 neu-oncogene.

4. Liposomal Transfection With IFIX to Treat Humans

Based on the results of the in vivo animal studies described in U.S. Pat. No. 5,641,484, those of skill in the art will understand and predict the enormous potential for human treatment of HER2/neu-mediated cancers with IFIX DNA complexed to liposomes. Clinical studies to demonstrate these effects are contemplated. Those of skill in the art will recognize that the best treatment regimens for using IFIX to suppress HER2/neu-mediated cancers can be straightforwardly determined. This is not a question of experimentation, but rather one of optimization, which is routinely conducted in the medical arts. In vivo studies in nude mice provide a starting point from which to begin to optimize the dosage and delivery regimes. The frequency of injection is initially once a week, as was done in the mice studies described in U.S. Pat. No. 5,641,484. However, this frequency might be optimally adjusted from one day to every two weeks to monthly, depending upon the results obtained from the initial clinical trials and the needs of a particular patient. Human dosage amounts can initially be determined by extrapolating from the amount of IFIX used in mice, approximately 15 μg of plasmid DNA per 50 g body weight. Based on this, a 50 kg woman would require treatment with 15 mg of DNA per dose. Of course, this dosage amount may be adjusted upward or downward, as is routinely done in such treatment protocols, depending on the results of the initial clinical trials and the needs of a particular patient. These clinical trials are anticipated to show utility of IFIXT33D, S35D, and/or T33DS35D and other neu-suppressing gene products for the treatment of HER2/neu-overexpressing cancers in humans. Dosage and frequency regimes will initially be based on the data obtained from in vivo animal studies, as is done frequently in the art.

H. Electroporation

In certain embodiments of the present invention, the expression construct is introduced into the cell via electroporation. Electroporation involves the exposure of a suspension of cells and/or DNA to a high-voltage electric discharge.

Transfection of eukaryotic cells using electroporation has been quite successful. Mouse pre-B lymphocytes have been transfected with humankappa-immunoglobulin genes (Potter et al., 1984), and/or rat hepatocytes have been transfected with the chloramphenicol acetyltransferase gene (Tur-Kaspa et al., 1986) in this manner.

I. Calcium Phosphate and/or DEAE-Dextran

In other embodiments of the present invention, the expression construct is introduced to the cells using calcium phosphate precipitation. HumanKB cells have been transfected with adenovirus 5 DNA (Graham and Van Der Eb, 1973) using this technique. Also in this manner, mouse L(A9), mouse C127, CHO, CV-1, BHK, NIH3T3 and/or HeLa cells were transfected with a neomycin marker gene (Chen and Okayama, 1987), and/or rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and/or erythroleukemia cells (Gopal, 1985).

J. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and/or enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten and/or gold beads.

K. Direct Microinjection and/or Sonication Loading

Further embodiments of the present invention include the introduction of the expression construct by direct microinjection and/or sonication loading. Direct microinjection has been used to introduce nucleic acid constructs into Xenopus oocytes (Harland and Weintraub, 1985), and/or LTK-fibroblasts have been transfected with the thymidine kinase gene by sonication loading (Fechheimer et al., 1987).

L. Adenoviral Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

V. Combination Treatments

In order to increase the effectiveness of a form of IFIX, or expression construct coding therefore, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that IFIX gene therapy could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

A skilled artisan recognizes that in addition to the IFIX forms described herein for the purpose of inhibiting cell growth, other chemotherapeutic agents are useful in the treatment of neoplastic disease. Examples of such chemotherapeutic agents are described in the following table.

TABLE 3
Chemotherapeutic Agents Useful In Neoplastic Disease

| | | NONPROPRIETARY | |
|---|---|---|---|
| | TYPE OF | NAMES | |
| CLASS | AGENT | (OTHER NAMES) | DISEASE |
| Alkylating Agents | Nitrogen Mustards | Mechlorethamine | Hodgkin's disease, |
| | | (HN₂) | non-Hodgkin's lymphomas |
| | | Cyclophosphamide | Acute and chronic |
| | | Ifosfamide | lymphocytic leukemias, |
| | | | Hodgkin's disease, |
| | | | non-Hodgkin's |
| | | | lymphomas, multiple |
| | | | myeloma, |
| | | | neuroblastoma, breast, |
| | | | ovary, lung, Wilms' |
| | | | tumor, cervix, testis, |
| | | | soft-tissue sarcomas |
| | | Melphalan | Multiple myeloma, breast, |
| | | (L-sarcolysin) | ovary |
| | | Chlorambucil | Chronic lymphocytic |
| | | | leukemia, primary |
| | | | macroglobulinemia, |
| | | | Hodgkin's disease, |
| | | | non-Hodgkin's lymphomas |
| | Ethylenimenes and | Hexamethylmelamine | Ovary |
| | Methylmelamines | Thiotepa | Bladder, breast, ovary |
| | Alkyl Sulfonates | Busulfan | Chronic granulocytic |
| | | | leukemia |
| | Nitrosoureas | Carmustine (BCNU) | Hodgkin's disease, |
| | | | non-Hodgkin's lymphomas, |
| | | | primary brain tumors, |
| | | | multiple myeloma, malignant |
| | | | melanoma |
| | | Lomustine (CCNU) | Hodgkin's disease, |
| | | | non-Hodgkin's lymphomas, |
| | | | primary brain tumors, |
| | | | small-cell lung |
| | | Semustine | Primary brain tumors, |
| | | (methyl-CCNU) | stomach, colon |
| | | Streptozocin | Malignant pancreatic |
| | | (streptozotocin) | insulinoma, malignant |
| | | | carcinoid |
| | Triazines | Dacarbazine (DTIC; | Malignant melanoma, |
| | | dimethyltriazenoimidazolecarboxamide) | Hodgkin's disease, soft-tissue |
| | | | sarcomas |
| Antimetabolites | Folic Acid | Methotrexate | Acute lymphocytic |
| | Analogs | (amethopterin) | leukemia, choriocarcinoma, |
| | | | mycosis fungoides, breast, |
| | | | head and neck, lung, |
| | | | osteogenic sarcoma |
| | Pyrimidine | Fluouracil | Breast, colon, stomach, |
| | Analogs | (5-fluorouracil; 5-FU) | pancreas, ovary, head and |
| | | Floxuridine | neck, urinary bladder, |
| | | (fluorode-oxyuridine; | premalignant skin lesions |
| | | FUdR) | (topical) |
| | | Cytarabine (cytosine | Acute granulocytic and |
| | | arabinoside) | acute lymphocytic leukemias |
| | Purine Analogs | Mercaptopurine | Acute lymphocytic, acute |
| | and Related | (6-mercaptopurine; | granulocytic and chronic |
| | Inhibitors | 6-MP) | granulocytic leukemias |
| | | Thioguanine | Acute granulocytic, acute |
| | | (6-thioguanine; TG) | lymphocytic and chronic |
| | | | granulocytic leukemias |
| | | Pentostatin | Hairy cell leukemia, mycosis |
| | | (2-deoxycoformycin) | fungoides, chronic |
| | | | lymphocytic leukemia |
| Natural Products | Vinca Alkaloids | Vinblastine (VLB) | Hodgkin's disease, |
| | | | non-Hodgkin's lymphomas, |
| | | | breast, testis |
| | | Vincristine | Acute lymphocytic |
| | | | leukemia, neuroblastoma, |
| | | | Wilms' tumor, |
| | | | rhabdomyosarcoma, |
| | | | Hodgkin's disease, |
| | | | non-Hodgkin's lymphomas, |
| | | | small-cell lung |
| | Epipodophyllotoxins | Etoposide | Testis, small-cell lung and |
| | | Tertiposide | other lung, breast, Hodgkin's |
| | | | disease, non-Hodgkin's |
| | | | lymphomas, acute |
| | | | granulocytic leukemia, |
| | | | Kaposi's sarcoma |
| | Antibiotics | Dactinomycin | Choriocarcinoma, Wilms' |
| | | (actinomycin D) | tumor, rhabdomyosarcoma, |
| | | | testis, Kaposi's sarcoma |
| | | Daunorubicin | Acute granulocytic and |
| | | (daunomycin; | acute lymphocytic leukemias |
| | | rubidomycin) | |
| | | Doxorubicin | Soft-tissue, osteogenic and |
| | | | other sarcomas; Hodgkin's |
| | | | disease, non-Hodgkin's |
| | | | lymphomas, acute leukemias, |
| | | | breast, genitourinary, |
| | | | thyroid, lung, stomach, |
| | | | neuroblastoma |
| | | Bleomycin | Testis, head and neck, skin, |
| | | | esophagus, lung and |
| | | | genitourinary tract; |
| | | | Hodgkin's disease, |
| | | | non-Hodgkin's lymphomas |
| | | Plicamycin | Testis, malignant |
| | | (mithramycin) | hypercalcemia |
| | | Mitomycin (mitomycin | Stomach, cervix, colon, |
| | | C) | breast, pancreas, bladder, |
| | | | head and neck |
| | Enzymes | L-Asparaginase | Acute lymphocytic leukemia |
| | Biological | Interferon alfa | Hairy cell leukemia., |
| | Response | | Kaposi's sarcoma, |
| | Modifiers | | melanoma, carcinoid, renal |
| | | | cell, ovary, bladder, |
| | | | non-Hodgkin's lymphomas, |
| | | | mycosis fungoides, multiple |
| | | | myeloma, chronic |
| | | | granulocytic leukemia |
| Miscellaneous | Platinum | Cisplatin (cis-DDP) | Testis, ovary, bladder, head |
| Agents | Coordination | Carboplatin | and neck, lung, thyroid, |
| | Complexes | | cervix, endometrium, |
| | | | neuroblastoma, osteogenic |
| | | | sarcoma |
| | Anthracenedione | Mitoxantrone | Acute granulocytic |
| | | | leukemia, breast |
| | Substituted Urea | Hydroxyurea | Chronic granulocytic |
| | | | leukemia, polycythemia vera, |
| | | | essental thrombocytosis, |
| | | | malignant melanoma |
| | Methyl Hydrazine | Procarbazine | Hodgkin's disease |
| | Derivative | (N-methylhydrazine, | |
| | | MIH) | |
| | Adrenocortical | Mitotane (o,p′-DDD) | Adrenal cortex |
| | Suppressant | Aminoglutethimide | Breast |
| Hormones and | Adrenocorti | Prednisone (several | Acute and chronic |
| Antagonists | costeroids | other equivalent | lymphocytic leukemias, |
| | | preparations available) | non-Hodgkin's lymphomas, |
| | | | Hodgkin's disease, breast |
| | Progestins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone | Breast |
| | | propionate | |
| | | Fluoxymesterone (other | |
| | | preparations available) | |
| | Antiandrogen | Flutamide | Prostate |
| | Gonadotropin- | Leuprolide | Prostate |
| | releasing | | |
| | hormone analog | | |

In addition to the chemotherapeutic agents listed above, any analog or derivative variant of the those listed are within the scope of the invention.

B. Radiotherapy

In addition to the IFIX forms described herein for the purpose of inhibiting cell growth, radation-based therapies are useful. That is, other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with, for example, Ad-IFIX gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a secondary gene therapy in which a second therapeutic polynucleotide is administered before, after, or at the same time a first therapeutic polynucleotide encoding all of part of a wildtype or altered, such as mutant, form of IFIX. Delivery of a vector encoding either a full length or truncated mutant form of IFIX in conjuction with a second vector encoding one of the following gene products will have a combined anti-hyperproliferative effect on target tissues. Alternatively, a single vector encoding both genes may be used. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{Waf1/Cip1}, and p27^{KIP1}. The p16^{INK4} gene maps to a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, Bik/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VI. Pharmaceutical Preparations

Pharmaceutical compositions of the present invention comprise an effective amount of one or more forms of IFIX or additional agent dissolved or dispersed in a pharmaceutically acceptable carrier or excipient. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one IFIX form or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifingal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The IFIX form may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The IFIX form may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the IFIX form is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fingi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

VII. Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods

Cell Lines and Plasmids

MCF-10A and MCF-12A cells were maintained in DMEM/F12 media containing 5% horse serum, 10 μg/ml bovine insulin, 20 ng/ml epidermal growth factor, 160 ng/ml cholera toxin, 0.5 μg/ml hydrocortisone, and 250 ng/ml fungizone. Daudi, Raji, HL-60, and U937 cells were grown in RPMI medium containing 10% fetal bovine serum. All other cell lines were cultured in DMEM/F12 media containing 10% fetal bovine serum. The IFIX expression vector, CMV-IFIX, was constructed by inserting an IFIXα1 cDNA fragment into pCMV-Tag2B (Flag) (Stratagene; La Jolla, Calif.). To generate IFIX stable cell lines, CMV-IFIX was transfected into MDA-MB-468 or MCF-7 cells. After 3 weeks of G418 selection (500 μg/ml), the G418-resistant colonies were screened for IFIX expression by western blot using an anti-Flag antibody (M5, Sigma, St. Louis, Mo.). Control derivatives of MDA-MB-468 and MCF-7 that carry pCMV-Tag2B were similarly established.

Determination of Gene Expression

The expression of IFIX in cell lines was determined by using Northern blot analysis performed as previously described (Wen et al., 2001). The expression of glyceraldehyde-3-phosphate dehydrogenase (GAPDH) was used a control for RNA loading. The IFIX mRNA levels on the Matched Tumor/Normal Expression Array (Clontech, Palo Alto, Calif.), which is a nylon membrane spotted with paired cDNA from tumor and normal tissues of individual patients, was determined by hybridization with an IFIX cDNA probe. As a control for RNA loading, the ubiquitin mRNA level was also determined. RT-PCR was used to determine the expression of IFIX in freshly collected normal and breast cancer tissues from five patients with various stages of breast cancer including one with only ductal carcinoma in situ. Total RNA was isolated from tissues using Atlas Pure Total RNA Labeling System (Clontech; Palo Alto, Calif.) and reverse transcription was performed using SuperScript First-Strand Synthesis System (Invitrogen, Carlsbad, Calif.). PCR was performed for 35 cycles at 94° C. for 40 sec, 56° C. for 1 min, and 72° C. for 40 sec. Primers 5′-GGAACAGAGTCAGCATCC-3′ (SEQ ID NO:10) and 5′-CTGCTGGATGGCGGTTGG-3′ (SEQ ID NO: 11) were used to amplified a 224 bp fragment of IFIX□. As a control for the quality of RNA samples, a 600 bp GAPDH cDNA fragment was amplified using primers 5′-TGAAGGTCGGAGTCAACGGA-3′ (SEQ ID NO:12) and 5′-GGCATGGACTGTGGTCATGA-3′ (SEQ ID NO: 13).

Determination of Growth, In Vitro Transformation and In Vivo Tumorigenicity of Breast Cancer Cells

The 3-(4,5-dimethylthiazol-2-yl)-2.5-diphenyltetrazolium bromide (MTT) and the soft agar assays were used to determine the anchorage-dependent and -independent, respectively, in vitro cell growth and were performed as previously described (Wen et al., 2001; Shao et al., 1997). To determine the tumorigenicity, 1×10⁶ cells were injected into a mammary fat pad (MFP) of 6-week old female nude mice and the growth of tumors was monitored weekly. For each experiment, a cell line was injected into 3 mice with each mouse injected at 2 MFP.

IFIX Gene Therapy

One million MDA-MB-468 cells in 200 μl of phosphate-buffered saline (PBS) were injected into a MFP of 4-5-week old female nude mice. Each cell line was injected into 10 mice with each mouse injected at 2 MFP. After tumors grew to 0.5 cm in diameter, mice were treated twice a week by intratumoral injection. Tumor-bearing mice were randomly divided into two treatment groups with each tumor injected with 22.5 μg of the liposome SN2 in 50 μl of PBS (Zou et al,. 2002) mixed with 15 μg of either CMVIFIX or the control vector pCMV-Tag2B.

Histone H1 Kinase Assay

Cells were lysed with RIPA-B Buffer (20 mM Na₂PO₄ (pH 7.4), 150 mM NaCl, 1% Triton X-100, 100 mM NaF, 2 mM Na₃VO₄, 5 mM phenylmethylsulfonyl fluoride, 1% aprotinin). Lysate containing 200-400 μg of protein was incubated at 4° C. for 1 hr with 2 μg of anti-CDK2 antibody (Santa Cruz Biotechnology, Inc. Santa Cruz, Calif.) or 1.5 μg anti-p34^{CDC2} antibody (Santa Cruz Biotech.) followed by incubation with Protein A-agarose for 2 hrs. The immunoprecipitates were washed twice with PBS, once with kinase buffer (10 mM Tris [pH 7.4], 150 mM NaCl, 10 mM MgCl₂, and 0.5 mM dithiothreitol), and then resuspended in 40 μl of kinase buffer containing 2 μg of histone H1 (Sigma), 25 μM ATP, and 5 μM γ-³²P ATP. The kinase reaction was terminated by adding 40 μl of SDS-PAGE loading buffer after a 15 min incubation at room temperature (CDK2) or 30 min incubation at 30° C. (p34^{CDC2}). Samples were resolved by SDS-PAGE and the phosphorylated histone H1 was visualized by autoradiography.

### Example 2

### IFIX is a Novel Human HIN-200 Gene

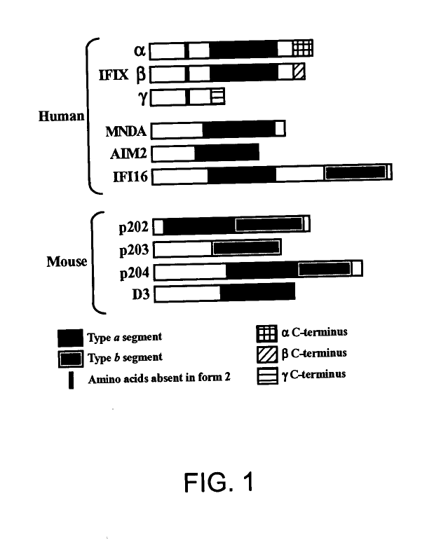

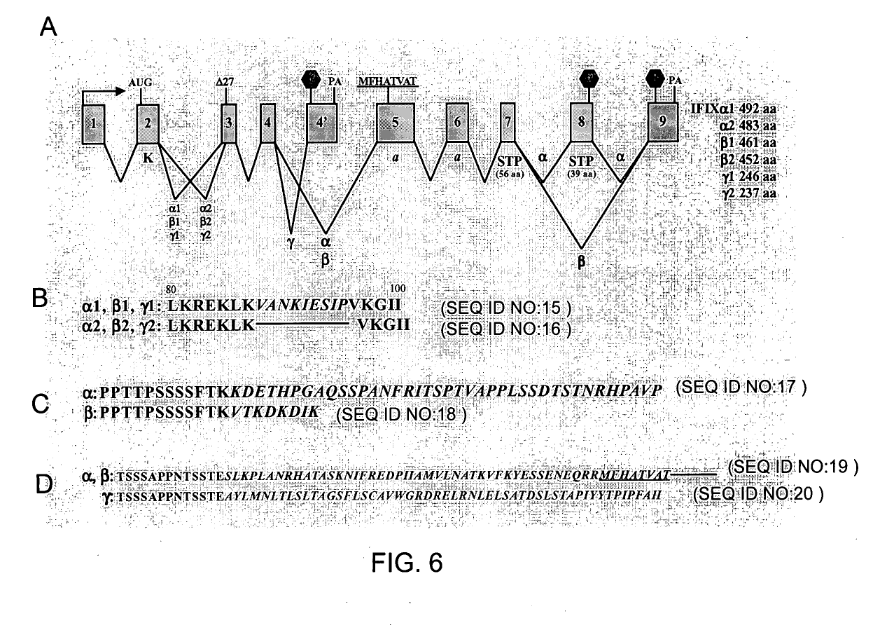

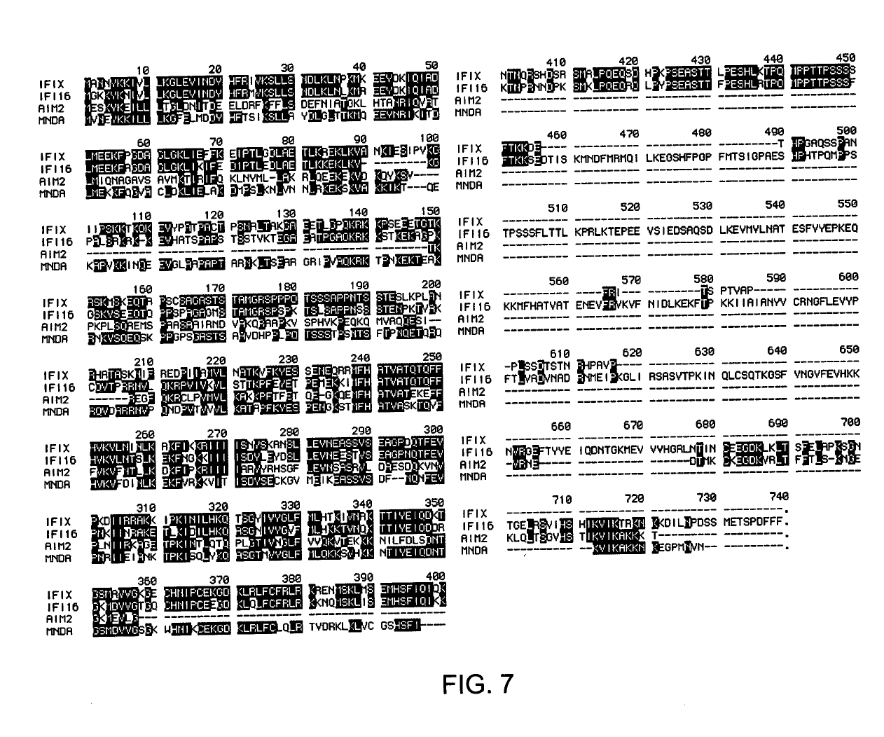

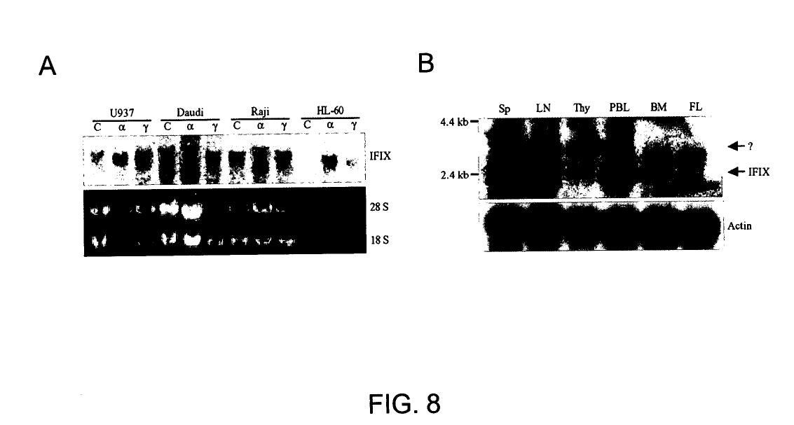

To identify the potential human counterpart of p202, the inventors used the amino acid sequence of p202a to query human-specific nr, est and htgs databases at the National Center for Biotechnology Information by using the tblastn protocol. These queries identified a new gene IFIX in addition to three previously known human HIN-200 family members, MNDA, IFI16, and AIM2. The IFIX gene is located between MNDA and IFI16 on the chromosome 1 q21-23. The IFIX cDNAs were obtained by RT-PCR using total RNA isolated from IFN-α-treated Daudi cells. Each cDNA clone was confirmed by DNA sequencing. The inventors identified six alternatively spliced IFIX isoforms (α1, α2, β1, β2, γ1, and γ2) based on the comparison between cDNA sequences and the genomic sequence (FIG. 1 and FIG. 6a). IFIXα2, β2, and γ2 contain identical, naturally occurring 9-amino acids deletion in the N-terminal domain (FIG. 6b). The α and β forms contain a type a 200-amino acid signature motif of HIN-200 proteins, whereas the γ forms do not have this motif (Johnstone and Trapani, 1999). The C-termini of α, β, and γ isoforms are diverse due to alternative splicing (FIG. 6c, 6d). The longest isoform of IFIX is α1, which contains 492 amino acids with an apparent molecular weight of ˜53 kD. Because IFIXα1 possesses the most structural features among these isoforms, the inventors performed most functional studies using IFIXα1. Unless otherwise specified, IFIX refers to IFIXα1 in the Examples. The identity between the deduced IFIX amino acid sequence and other members of human HIN-200 family are: IFI16, 67%; MNDA, 53%; and AIM2, 31% (FIG. 7). IFIX is unlikely the human homolog of p202a because the similarity between amino acid sequences of IFIX and p202a, is limited to the HIN-200 signature motif IFIX contains a putative nuclear localization signal, PQKRK (amino acids 136-140), in the N-terminal domain shared by most family members (Johnstone and Trapani, 1999) (FIG. 7). The IFIX mRNA level is characteristically induced by IFN-α and IFN-γ in several human cancer cell lines of hematopoietic origin (FIG. 8a). A tissue distribution study showed that IFIX mRNA (2.4 kb) is readily detected in spleen, lymph node, and peripheral blood leukocyte, but to a lesser extent in thymus, bone marrow, and fetal liver (FIG. 8b). No detectable level of IFIX mRNA was found in adult brain, heart, skeletal muscle, colon, kidney, liver, small intestine, placenta and lung. These results indicate that IFIX expression is involved in immune response. Based on the chromosomal location, sequence and structural homology, and IFN-inducibility (Lengyel et al., 1995; Johnstone and Trapani, 1999), IFIX is concluded to be a novel member of the human HIN-200 family.

### Example 3

### IFIX is Down-Regulated in Human Breast Cancers

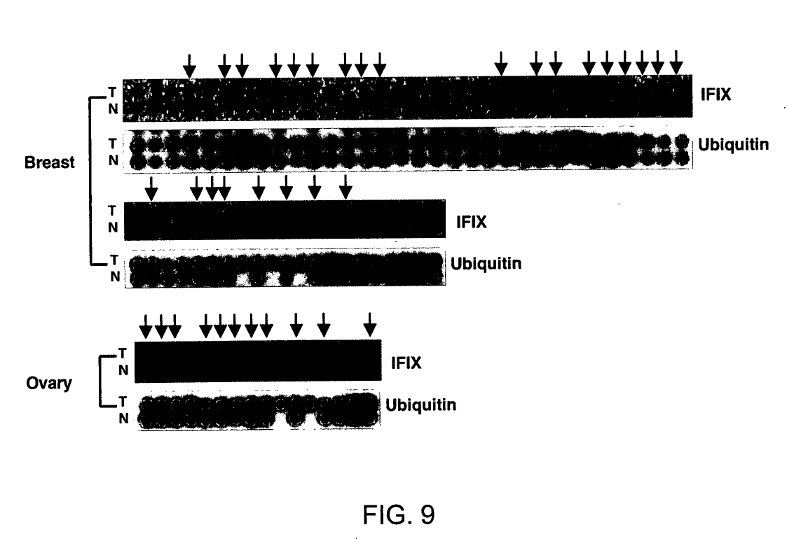

To investigate the possible altered expression of IFIX in human cancers, the inventors first examined the Matched Tumor/Normal Expression Array. The data showed IFIX expression is down-regulated in ˜52% (26/50) of advanced breast tumors and in 79% (11/14) of ovarian tumors. IFIX is also down-regulated in other tumor types to various degrees (FIG. 9).

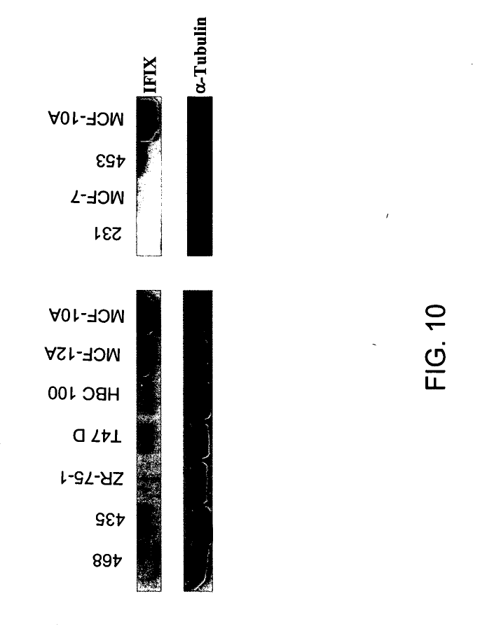

Specifically, the expression array was hybridized with IFIX cDNA probe and found that, after normalization with ubiquitin expression for loading control, IFIX expression is down-regulated in tumors from ovary (11/14, 79%), breast (27/50, 54%), prostate (2/4, 50%), and lung (10/21, 48%). Other tumors with IFIX downregulation include rectum (7/18, 39%), colon (11/34, 32%), kidney (7/20, 35%), thyroid (2/6, 33%), uterus (12/42, 29%), and stomach (9/27, 33%). This result indicates that IFIX is downregulated in many human cancers, specifically in breast and ovarian cancers. To further examine this correlation, a panel of breast cancer cell lines was screened and the “normal” breast cell lines for IFIX expression by western blot using IFIX-specific antibody. As shown in FIG. 10, consistent to the expression array data, IFIX expression is readily detectable in “normal” immortalized breast cell lines such as MCF-10A and MCF-12A. In contrast, in breast cancer cell lines, IFIX is either downregulated (e.g., MDA-MB-435) or undetectable (e.g., MDA-MB-468, ZR-75-1, T47D, HBC 100, MDA-MB-231, MCF-7, and MDA-MB-453).

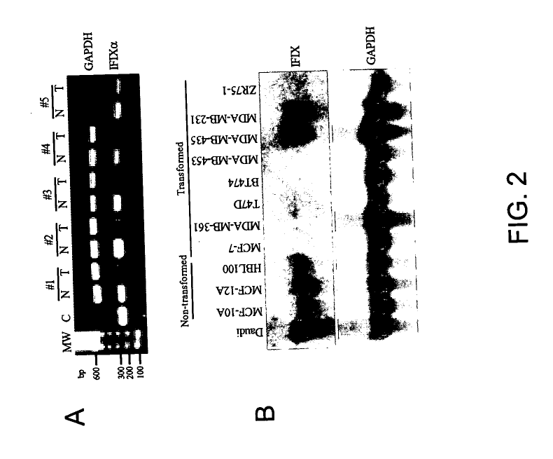

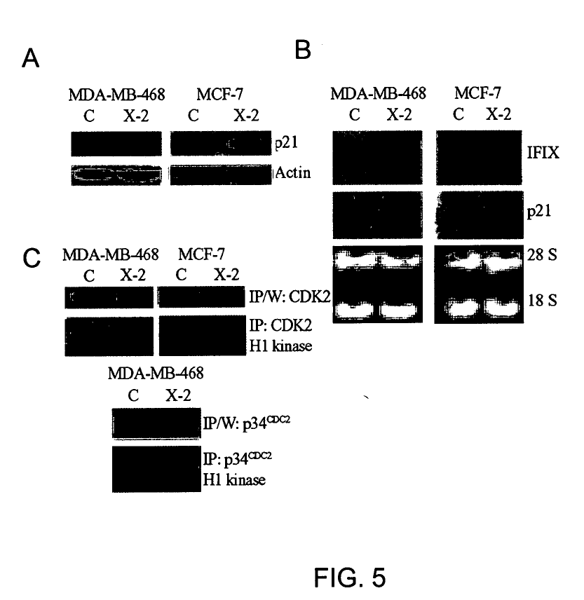

To confirm that the expression of IFIX was reduced in breast cancer, IFIX mRNA levels were examined in matched normal and tumor tissues collected from five breast cancer patients using RT-PCR. The expression of IFIX was detected in all tissues examined, however, the level of IFIX in tumor was lower than that in normal tissue in each matched pair (FIG. 2a). The inventors also examined the expression of IFIX in a panel of human breast epithelial cell lines. IFIX expression was detected in all three non-tumorigenic cell lines. In contrast, 7 out of 9 breast cancer cell lines did not express detectable IFIX (FIG. 2b and 5b). These data show that the expression of IFIX is reduced in breast cancer and indicate that IFIX functions as a tumor suppressor.

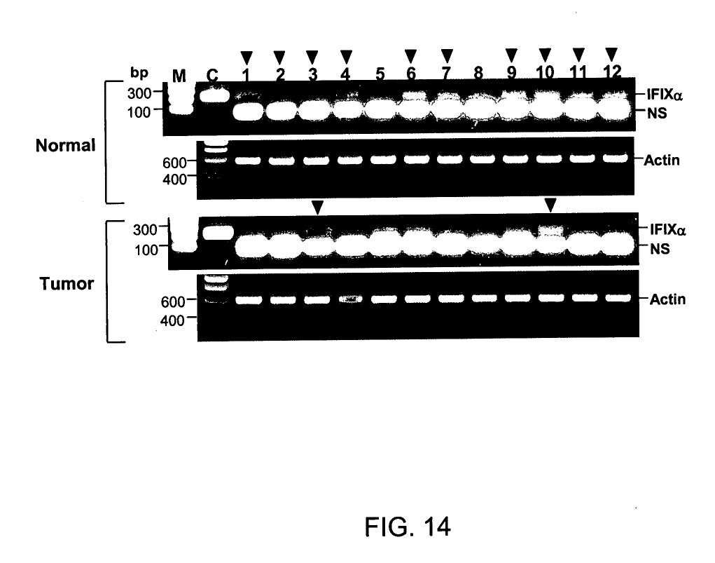

Additional data indicating that IFIX is down-regulated in human breast cancers is provided in FIG. 14. As shown therein, As shown in FIG. 14, IFIXα expression is detectable in 10 out of 12 normal breast tissue samples. In contrast, only 2 out of 12 breast carcinoma tissues have detectable IFIXα expression. This result indicates that IFIX is downregulated in breast tumors.

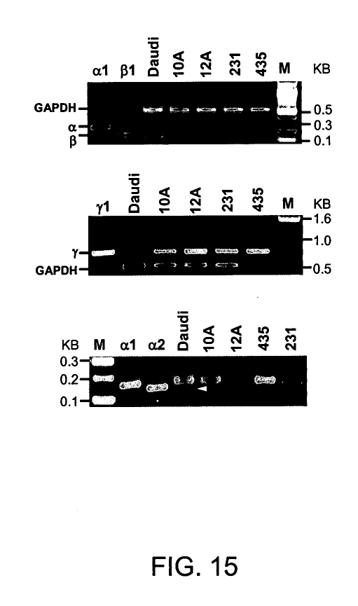

To determine the identity of IFIX isoforms in the IFIX-expressing cell lines, the present inventors performed RT-PCR using specific primers for these isoforms. As shown in FIG. 15 (top and middle panels), the IFIXα, β, and γ isoforms are present in these cell lines, although the 27-bp deletion in the “form 2” isoforms cannot be distinguished at this gel resolution. To further determine the presence of the “form 2” isoforms, the present inventors designed primers that flank the A27 region (FIGS. 6 and 7) followed by RT-PCR. Consistent with the fact that the “form 2” isoforms were isolated from Daudi cells, the expression of “form 2” isoforms is detectable in Daudi cells but the expression levels are much lower than that of the “form 1” isoforms with the 27-bp region (FIG. 14, bottom panel). However, under the present experimental conditions, the “form 2” isoforms appeared to be not expressed or undetectable in other IFIX-expressing cell lines.

### Example 4

### IFIX Suppresses Breast Cancer Cell Growth and Tumorigenicity

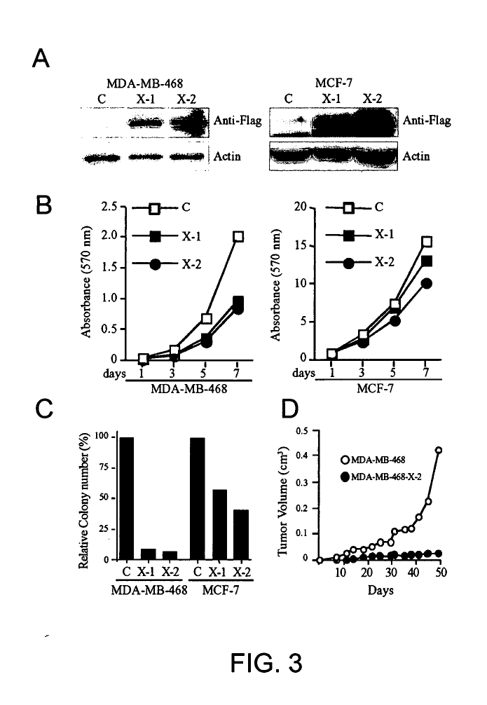

To further characterize the possible tumor suppressor function of IFIX, IFIX was stably expressed in two human breast cancer cell lines, MDA-MB-468 and MCF-7, that did not express endogenous IFIX (FIG. 2b, 3a, 5b). Examination of the growth rates of control cell lines and IFIX expressing cell lines showed that the expression of IFIX reduced the growth of breast cancer cells (FIG. 3b). The soft agar assay was used to determine the effect of IFIX on the in vitro transformation property. As shown in FIG. 3c, the number of foci of IFIX-expressing cell lines was reduced significantly as compared with their parental cells. This result indicated that IFIX was able to suppress the transformation phenotype of breast cancer cells and predicted a loss of tumorigenicity of IFIX-expressing breast cancer cells. Tumorigenicity of the IFIX stable cell lines was then tested by implanting MDA-MB-468 and an IFIX expressing derivative 468-X-2 into MFP of 6-week old female nude mice. As shown in FIG. 3d, MDA-MB-468 cells are highly tumorigenic, whereas the tumorigenicity of 468-X-2 cells is reduced.

In addition, in breast cancer cell lines, the expression of IFIX mRNA (SKBR3, MDA-MB-231, and MCF-7) and protein (MCF-7) is induced upon IFN treatment. Furthermore, both mouse and human HIN-200 gene cluster are clustered at chromosome 1q21-23 (Lengyel et al., 1995), and IFIX co-localizes with its human family members on chromosome 1q22. However, IFIX is not a human homolog of p202, since IFIX possesses only one 200-amino acid repeat, whereas p202 has both type a and type b repeats. In addition, IFIX does not have the N-terminal domain unique to p202 (Johnstone and Trapani, 1999).

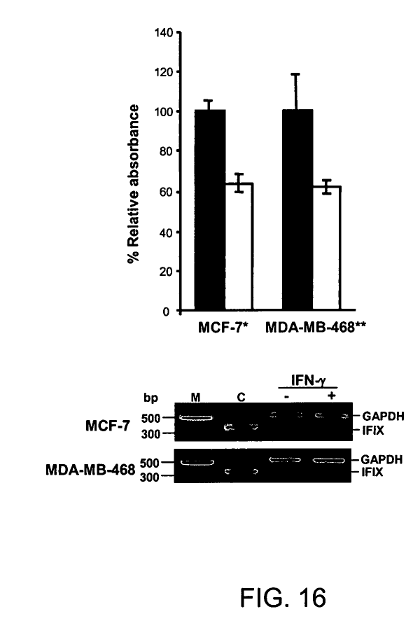

To characterize the role of IFIX as a tumor suppressor, two human breast cancer cell lines, MCF-7 and MDA-MB-468 that express very low levels of endogenous IFIX (FIGS. 2B, 5B, and 16) were employed. Consistent with a previous report (Gooch et al., 2000), IFN-γ treatment suppressed the growth of these breast cancer cells (FIG. 16, top panel), which correlated with the induction of IFIX (FIG. 16, bottom panel). Thus, this indicates that IFIX, such as the IFIXα1 isoform, is a major mediator of tumor suppressor activity of IFN.

Observations that IFIX suppresses growth, transformation, and tumorigenicity of breast cancer cell lines indicate IFIX possesses tumor suppressor activity.

### Example 5

### IFIX Treatment Results in Therapeutic Efficacy

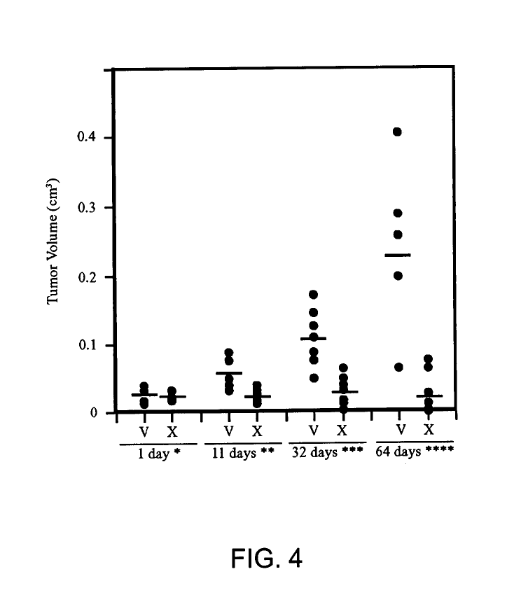

IFIX-based gene therapy was studied. Female nude mice were inoculated with MDA-MB-468 cells into their MFP and tumors were allowed to grow to about 0.5 cm in diameter. Tumors were injected with the liposome SN2 together with either CMV-IFIX or pCMV-Tag2B. SN2 was selected as the gene delivery system because it is a non-viral, stable liposome-forming cationic lipid formulation and has been proven to be highly efficient in gene delivery (Zou et al., 2002). As shown in FIG. 4, the CMV-IFIX/SN2 treatment yielded significant antitumor activity as compared with the pCMV-Tag2/SN2 treatment. The anti-tumor activity of IFIX demonstrated in this pre-clinical model indicates that IFIX is useful for cancer gene therapy, particularly breast cancer gene therapy.

### Example 6

### IFIX Up-Regulates P21^{CIP1}

IFN-induced growth arrest is known to be associated with an elevated level of the CDK inhibitor (CKI), p21^{CIP1} (Zhou et al., 2002; Naldini et al., 2002; Matsuoka et al., 1998). Because the expression of IFIX is induced by IFN (FIG. 8a), we therefore investigated the mRNA and protein levels of p21^{CIP1} in IFIX-stable transfectants. As shown in FIGS. 5a and 5b, both p21^{CIP1} protein and mRNA levels are up-regulated in IFIX-stable cell lines. However, there are no detectable changes in the expression of other CKIs, such as p27^{KIP1}, p57^{KIP2}, and p16^{INK4a} in IFIX-expressing derivatives.

Since p21^{CIP1} is a universal CKI, up-regulation of p21^{CIP1} should inactivate the kinase activity of CDK2 in IFIX-stable cells. Therefore, an immuno-complex kinase assay was used to compare the CDK2 kinase activity in IFIX stable cell lines and control cells. As expected, CDK2 activity is reduced in 468-X-2 and MCF-X-2, as compared to parental cells (FIG. 5c). Western blot shows that there are comparable amounts of CDK2 protein used in the kinase assay. However, since MDA-MB-468 cells do not possess functional pRb, inhibition of MDAMB-468 cell growth by IFIX cannot simply be attributed to the inactivation of CDK2 leading to G1/S phase arrest (MacLachlan et al., 1995). In one embodiment, p21^{CIP1} also inhibits p34_{CDC2}, a G2/M phase CDK (Yu et al,. 1998; Niculescu et al., 1998). The present inventors studied the p34_{CDC2} kinase activity in IFIX-expressing MDA-MB-468 cells and found that it was much lower than that of the control cells (FIG. 5c).

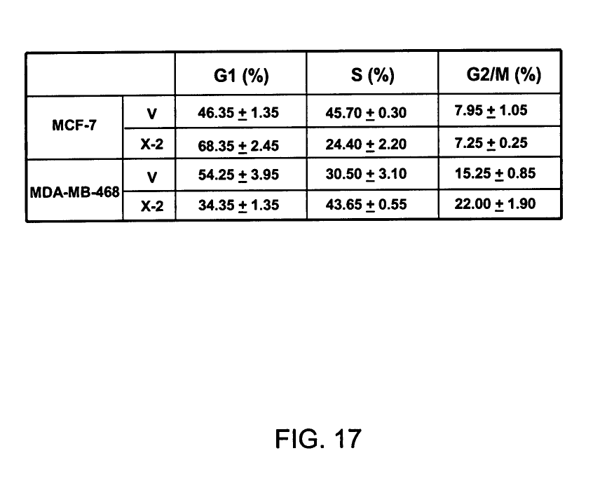

To further characterize this observation, the present inventors performed a flow cytometry analysis to determine any changes in the cell cycle distributions caused by the expression of IFIX. As shown in FIG. 17, a significant G1-phase accumulation and S-phase reduction was observed in MCF-X-2 cells. In contrast, 468-X-2 cells exhibited a significant S- and G2/M-phase accumulation. This observation not only provides an explanation for the slower growth rate of IFIX stable cell lines (FIG. 3B), but also correlates with the inactivation of Cdk2 leading to G1-phase accumulation in MCF-7 in which pRB/E2F pathway is intact and the inactivation of p34^{Cdc2} resulting in a blockage of G2/M-phase entry in MDA-MB-468 in which pRB/E2F pathway is defective. The data suggest that the p53/pRB-independent p21^{CIP1} up-regulation contributes to IFIXα1-mediated anti-tumor activity in breast cancer cells.

The data presented in this Example indicate that inactivation of p34^{CDC2} contributes to the IFIX-mediated growth inhibition in MDA-MB-468 cells. Thus, the p53-independent p21_{CIP1} up-regulation contributes to IFIX-mediated anti-tumor activity in cancer cells, such as breast cancer cells.

### Example 7

### Isolation of IFIX Promoter Sequences

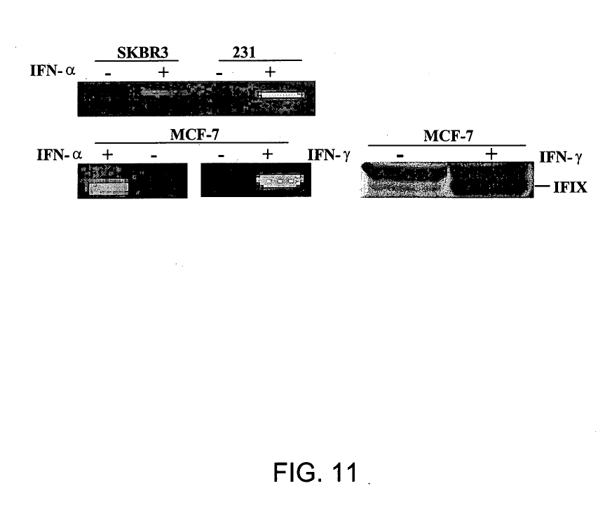

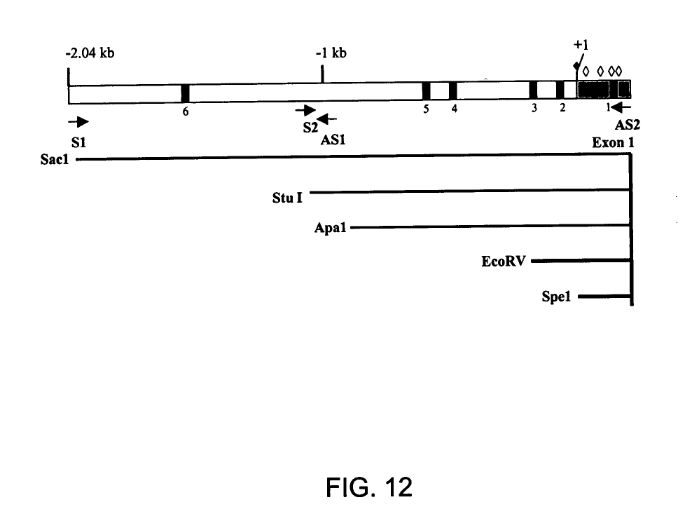

To study the regulation of IFIX transcription, the IFIX promoter was isolated. Using 5′ primer RACE kit (Clontech; Palo Alto, Calif.), the 5′ ends of IFIX mRNAs isolated from Daudi cells that express detectable level of IFIX mRNA were mapped (FIG. 11). Five potential transcriptional start sites were identified (FIG. 12). The 5′ untranslated region (5′UTR) includes exon 1 of IFIX gene. Two pairs of primers (S1/AS1 and S2/AS2) that overlap with each other were used to isolate a total ˜2 Kb promoter sequence. After verification by DNA sequencing, a computer search was performed for the putative trans-acting factor binding sites on the IFIX promoter. Several housekeeping transcriptional factor binding sites, e.g., SP1 and TATA, and regulatory factor binding sites, e.g., IFN-stimulated responsive elements (ISRE), STAT1, NF-κB, and estrogen receptor (ER) were identified. Five unique restriction enzyme sites were identified on the promoter that are useful for convenient construction of deletion mutants for promoter analysis.

### Example 7

### Determination of the Expression of IFIX in Human Breast Tumor Tissues

Studies may be performed determining IFIX expression is downregulated in human breast tumor tissues. Specifically, IFIX expression and the IFIX-mediated biological effect on the primary breast tumor tissues may be studied. Briefly, normal tissues and breast tumor samples may be separated into two portions: one portion may be quick frozen by liquid nitrogen for northern blot analysis; and the other portion may be fixed with formalin and embedded in paraffin. Immunohistochemical analysis of p202 protein expression was performed according to the protocol described previously (Xia et al., 1999). Both normal and tumor sections may be incubated with rabbit polyclonal antibody specific for IFIX followed by incubation with biotinylated rabbit anti-rabbit IgG, and subsequently with avidin-biotin-peroxidase before visualization. In a specific embodiment, the one identifies low IFIX expression (mRNA and protein) in tumors but not in the surrounding normal breast epithelial cells or tissues, e.g., skin.

### Example 8

### Determination of the Tumor Suppressor Activity of IFIX in Knockdown Cell Model

In the embodiment wherein IFIX functions as a tumor suppressor, normal breast cells will become cancerous when IFIX expression is downregulated. This may be studied by first generating the IFIX-knockdown (IFIX-kd) cell lines and then testing the potential tumorigenic phenotypes resulting from IFIX downregulation.

IFIX-kd cell lines may be generated by at least two approaches: (i) antisense (AS): the inventors have generated an expression vector in which a full-length IFIX antisense (IFIX-AS) is driven by CMV promoter. CMV-IFIX-AS may be transfected into MCF-10A and MCF-12A cells followed by G418 selection. The G418-resistant clones can be screened by western blot using anti-IFIX antibody. Since IFIX shares some sequence homology with other family members, i.e., IFI16, MNDA, and Aim2, in some embodiments the IFIX-AS clones lose expression of these proteins. To make sure that the IFIX-AS clones represent IFIX-kd cells, these clones may be screened for the expression of IFI16, MNDA, and Aim2. Western blot may be employed to detect IFIX and MNDA protein expression, since the antibodies against these two proteins are commercially available (Santa Cruz Biotechnology, Inc.; Santa Cruz, Calif.). Aim2 expression can be monitored by northern blot using Aim2 cDNA fragment as a probe. The clones that only lose IFIX protein expression can be subject to northern blot analysis to confirm the loss of IFIX mRNA in these clones; (ii) RNA interference (RNAi): RNAi technology represents a more specific gene knockdown strategy in mammalian cells than does the AS approach. Consequently, RNAi has been widely used to specifically knockout the expression of the gene of interest (Hannon, 2002; Paddison et al., 2002a; Paddison et al., 2002b; Sui et al., 2002). An RNAi expression cassette can be obtained. The inventors are in the process of constructing the RNAi vector (U6-IFIX) that should specifically target the unique region of IFIX (+465 AGACCTTGCTGAAACTCTT+483; SEQ ID NO:14) that shares no homology with the other three family members. Once the U6-IFIX may be made, one may transfect it into MCF-10A and MCF-12A followed by G418 selection. The screening and the confirmation of the IFIX-kd cells can be similar to that described above. In an alternative embodiment, the somatic knockout strategy using homologous recombination to generate knockout cells, e.g., p21^{CIP1} (Waldman et al., 1995) or Retinoid X Receptors (Chiba et al., 1997) may be utilized.

### Example 9

### Characterization of IFIX-KD Cells

In some embodiments of the present invention, the IFIX-AS and/or U6-IFIX expression will convert the normal breast cells, e.g., MCF-10A and MCF-12A, into breast cancer cells. Once the IFIX-kd cells become available, one may subject them to the in vitro growth assays, such as MTT and ³H-thymidine incorporation, to determine if the loss of IFIX expression would alter the cell growth rate. Since the expression of IFIX in breast cancer cells reduced the growth rates, in some embodiments IFIX-kd may have a faster growth rates than that of the parental control cells. To test whether the loss of IFIX expression would render the normal breast cells to exhibit transformation phenotype, one may seed the IFIX-kd cells and the parental control cells in the soft-agar. While MCF-10A and MCF-12A do not grow in soft-agar, one may see that the corresponding IFIX-kd cells will gain the ability to grow in soft-agar, i.e., the anchorage-independent growth, which is a typical transformation phenotype. If so, one may further test if these IFIX-kd cells are tumorigenic by inoculating the IFIX-kd cells and the parental control cells into the opposite mammary fat pads of the same female nude mice, namely, one side of the mouse inoculated with IFIX-kd cells and the other side the control cells. One may see that the IFIX-kd cells will become tumorigenic, but not the control cells.

### Example 10

### Determination of the Tumor Suppressor Activity of IFIX in Transgenic Mice Model

A typical approach to demonstrate the tumor suppressor activity of a candidate gene is by knocking out its mouse counterpart and then analyzing the resulting phenotype (if any) during tumorigenesis. However, in the case of IFIX, none of the four known mouse HIN-200 family members, p202, p203, p204, and D3 (Johnstone and Trapani, 1999), can be considered as the authentic mouse IFIX counterpart. Both p202 and p204 have type a and type b 200-amino acid repeats, and p203 has a type b repeat. Therefore, none of these three genes qualified to be the IFIX counterpart, since IFIX possesses only one type a repeat. Although D3 has a type a repeat, it lacks the serine/threine/proline-rich C-terminal domain present in IFIX protein. Thus, D3 should not be considered as the mouse counterpart of IFIX. Based on this structural consideration, the mouse IFIX is not known. Therefore, one can use two alternative yet exemplary approaches to demonstrate the tumor suppressor activity of IFIX in animal models.

In one embodiment, one may generate an IFIX transgenic mouse strain in which mouse mammary tumor epithelium specific promoter, such as mouse mammary tumor virus long terminal repeat (MMTV), will be used to direct IFIX gene expression. MMTV promoter has been shown to direct high-level expression of gene of interest to the mammary epithelium (Cardiff and Muller, 1993). An MMTV-IFIX expression vector may be constructed and the transgenic mice (in FVB/N genetic background) may be generated expressing IFIX. In some embodiments, one may obtain multiple strains of MMTV-IFIX mice with different level of IFIX expression in the mammary glands. RNA extracted from the mammary gland tissues of these strains can be verified and quantified for IFIX mRNA expression by real-time RT-PCR using IFIX-specific primers.

Although the normal human breast cells express IFIX, it is not known what effect IFIX overexpression will be on the mouse mammary glands. Thus, with the assistance from GEMF, one may examine the phenotype of the mammary glands of the MMTV-IFIX mice as compared with that of the control FVB/N mice. Especially before and after pregnancy, one may examine the mammary gland whole mounts during the process of involution. In some embodiments, IFIX may have a dose effect on the mammary glands. In that case, one may choose the mouse strain that has the minimum effect on the mammary gland, i.e., without gross abnormality, for the subsequent experiments. Given that, one may subject the female MMTV-IFIX mice (and the control FVB/N female mice without the transgene) to a carcinogenesis protocol in which the mice are treated with a chemical carcinogen, 7, 12-dimethylbenz[a]anthracene (DMBA) (Singer and Kusmierek, 1982), and a hormone, medroxyprogesterone acetate (MPA). A combination of these agents has been shown to cause predominantly mammary tumors in mice with relatively short latency (Aldaz et al., 1996). These mice (MMTV-IFIX and the control mice, 50 mice per group) may be MPA-supplemented (at 6-week old) before DMBA treatment (at 9, 10, 12, and 13-week old). MMTV-IFIX mice and the control mice without treatment (25 mice per group) serve as the control. The rate of tumorigenesis, tumor size, survival rate may be monitored. In the embodiment wherein IFIX functions as a tumor suppressor in mammary gland carcinogenesis by DMBA, one may see that in the DMBA treatment group MMTV-IFIX mice will exhibit an attenuated tumorigenesis as compared with that of the control FVB/N mice.

### Example 11

### Determination of Tumorigenesis of MMTV-IFIX and MMTV-NEU Hybrid Transgenic Mice

Another way to study the tumor suppressor function of IFIX is to see if IFIX can suppress the tumorigenesis of a transgenic mouse expressing an oncogene in mammary glands. For this purpose, one may chose MMTV-neu (it encodes an active neu oncogene with a single point mutation) oncomouse (Charles River Laboratories; Boston, Mass.) since HER-2/neu is known to be amplified/overexpressed in 20˜30% breast cancer patients (Slamon et al., 1987); and MMTV-neu transgenic mice are well characterized and have relatively short latency for tumorigenesis (100% by the age of 100 days after birth) (Muller et al., 1988; Guy et al., 1992). To determine whether the expression of IFIX could impede the neu-mediated tumorigenesis, one may cross MMTV-IFIX with MMTV-neu to generate offsprings carrying both transgenes, i.e., MMTV-IFIX/neu hybrid mice. The transgene (IFIX and neu) expression in mammary tissues may be examined by real-time RT-PCR. Once confirmed, one can compare the rate of tumorigenesis, tumor size, and survival rate in these three groups of mice, i.e., MMTV-neu, MMTV-IFIX, and MMTV-IFIX/neu. In the embodiment wherein IFIX functions as a tumor suppressor in the oncogene, e.g., neu-mediated tumorigenesis, one may see that MMTV-IFIX/neu mice will have a significant delay of tumorigenesis as compared with that of the MMTV-neu mice.

In additional embodiments of the present invention, at least one large retrospect study involving more patient samples is performed. One of skill in the art, based on the teachings provided herein and the knowledge in the art the appropriate methods and reagents to employ such a study. In a specific embodiment, the data to correlate IFIX expression with the different stages during the diseases progression is analyzed, preferably statistically. In particular, one may examine the correlation with lymph node metastasis. Given that IFIX expression is high in normal lymph node and IFIX functions as a tumor suppressor, in a particular embodiment IFIX expression may be downregulated in metastatic lymph node.

### Example 12

### Determination of the Mechanisms of IFIX Regulation

IFIX mRNA and protein expression was downregulated in human breast tumor tissues and in human breast cancer cell line, as shown herein. Although post-transcriptional control, e.g., mRNA stability, may be involved, in some embodiments the IFIX downregulation is on the transcriptional level (although it may also be post-transcriptional). Experiments may be performed to examine the transcriptional downregulation of IFIX in breast cancer cells. A panel of mutant IFIX promoters can be used to identify the cis-acting elements and the trans-acting factor that are responsible for the IFIX downregulation.

Although the normal breast cell lines, e.g., MCF-10A and MCF-12A, have detectable IFIX protein expression but not in the breast cancer cell lines, as shown herein, the IFIX mRNA expression is studied for correlation with the differential IFIX protein expression. One may perform a northern blot analysis using IFIX cDNA as a probe, for example, to examine the IFIX mRNA expression pattern between normal and cancer cells. Since the cDNA array data suggest that IFIX mRNA is downregulated in the breast tumor tissues, in some embodiments IFIX downregulation in cancer cells is on the transcriptional level. One may employ a nuclear run-on assay that is commonly used to monitor the transcriptional rate of a given gene (Derman et al., 1981). Briefly, the nuclei isolated from either normal cells, e.g., MCF-10A and MCF-12A, or the cancer cells, e.g., MCF-7, and MDA-MB-231, may be incubated with ribonucleotides (ATP, CTP, GTP, and ³²P-UTP) in the reaction buffer. The nascent RNA may be elongated only for one cycle. The total RNAs can then be isolated and serve as probes. The denatured IFIX cDNA along with the denatured controls, e.g., empty vector DNA and glyceraldehydes-3-phosphate dehydrogenase (GAPDH) cDNA, may be immobilized on Hybond N membranes. The radiolabeled RNAs may be hybridized with the membranes. The specific radiolabeled IFIX mRNA may be hybridized with IFIX cDNA on the membrane. The intensity of the IFIX-specific signal is indicative of the density of RNA polymerase II on the IFIX gene, and thus promoter activity. In the embodiment wherein IFIX transcriptional activity is higher in normal cells than in cancer cells, one may see that the nuclear run-on experiment will show a higher IFIX-specific signal using ³²P-RNA isolated from the normal cells than that from the cancer cells. In the embodiment wherein there were detectable IFIX-specific signals without significant difference between normal and cancer cells, the post-transcriptional regulation, e.g., RNA stability, may be involved. Ordinarily, the RNA stability study in the cells in the presence of pan-transcription inhibitor, e.g., actinomycin D, can be performed. However, since there is no detectable steady state IFIX mRNA in the cancer cells such as MCF-7 and MDA-MB-468, in one embodiment the stability of IFIX mRNA may be difficult to measure. Studies may be performed to characterize the mechanisms of IFIX downregulation in breast cancer cells in the embodiment wherein it is due to transcriptional downregulation.

### Example 13

### Comparison of the IFIX Promoter Activity Between Normal Breast and Breast Cancer Cells

One may transfect the IFIX promoter (2 kb)-driven luciferase expression vector, i.e., IFIX-luc, into the normal cells, e.g., MCF-10A and MCF-12A, and the cancer cells, e.g., MCF-7, and MDA-MB-231. pRL-TK may be be co-transfected as an internal transfection efficiency control using the Dual Luciferase Assay Kit (Promega). In the embodiment wherein IFIX is downregulated on the transcriptional level in cancer cells, one may see, after normalization, IFIX-luc transfection results in higher relative luc activity in normal cell lines than in the cancer cells.

### Example 14

### Identification of the Cis-Acting Element on IFIX Promoter Responsible for IFIX Transcriptional Downregulation in Breast Cancer Cells

To identify the cis-acting element responsible for the promoter silencing, one may use a panel of IFIX promoter deletion mutants and test their luc activity in the normal and cancer cells. The luc activity of the mutant promoter that yields no difference between normal and cancer cells is the candidate cis-acting element responsible for IFIX downregulation, in specific embodiments. This element may be referred to as TSE (Tumor Suppression Element). At least two possible embodiments depend on the nature of the differential regulation. In the first embodiment, if TSE interacts with a transcriptional activator that is missing in cancer cells, then one may see that the luc activity of the mutant promoter without TSE in normal cells reduces to the same level as that in cancer cells. In a second embodiment, if TSE interacts with a transcriptional repressor that is present in cancer cells, one may see a derepression of the mutant promoter without TSE in cancer cells. In that case, the luc activity of the mutant promoter without TSE in cancer cells may increase to that in the normal cells. To further characterize the enhancer or repressor function of TSE, one may subclone TSE and mutant TSE (mTSE) into a heterologous promoter, e.g., pGL2-promoter vector (Promega) that contains a SV40 minimum promoter, to generate pGL2pr-TSE-Luc and the control, pGL2pr-mTSE-Luc. One may then transfect these vectors into normal and cancer cells. In the embodiment wherein TSE interacts with a transcriptional activator present in normal cells but not in cancer cells, one may see that TSE will enhance the SV40 promoter activity of pGL2pr-TSE-Luc in normal cells but not in cancer cells. Again, in the embodiment wherein TSE interacts with a transcriptional repressor only present in the cancer cells, one may see that the SV40 promoter activity of pGL2pr-TSE-Luc is suppressed only in cancer cells but not in normal cells. In both cases, the control mTSE should not affect the SV40 promoter activity in normal or cancer cells.

### Example 15

### Identification of the Trans-Acting Factor on IFIX Promoter Responsible for IFIX Transcriptional Downregulation in Breast Cancer Cells

To identify the trans-acting factor that interacts with TSE, one may employ gel-shift assay. One may use ³²P-labeled TSE and mTSE oligonucleotides as probes and incubate them with nuclear extract isolated from normal or cancer cells in a gel-shift assay. One may perform competition assay with cold TSE or mTSE oligonucleotide to identify TSE-specific DNA/protein complex. In a specific embodiment, one may see the specific DNA/protein complex present (or absent) in normal cells but not in cancer cells. If TSE is a known trans-acting factor binding site (the computer search is available in many academic web sites, e.g. EMBL) and the antibody is commercially available, one may perform super-shift assay by incubating the specific antibody against the known trans-acting factor in nuclear extract isolated from normal or cancer cells. One may see that the specific antibody will either interact with the TSE-specific DNA/protein complex causing it to migrate slowly (super-shift) or it will abolish the complex, depending on where the antibody binds. In either case, in these embodiments it is expected to be a normal (or cancer) cell-specific phenomenon.

In the embodiment wherein the TSE-binding protein (TSEBP) is unknown, several methods may be used to clone the gene encoding the TSE-binding protein. One of the easier and quicker methods as compared to protein purification is the yeast one-hybrid screen (MATCHMAKER, one-hybrid system; Clontech; Palo Alto, Calif.), in which multiple copies of TSE can be placed 5′ to a minimum promoter-driven reporter gene, e.g., HIS3 or lacZ. The recombinant plasmid may then be transfected into yeast to establish at least one stable yeast strain. If the TSE-binding transcriptional activator is present in normal cells, one may then transform the stable yeast strain with a cDNA library derived from normal mammary glands (e.g., human mammary gland MATCHMAKER cDNA library; Clontech; Palo Alto, Calif.) in which the encoded proteins are synthesized as fusions with a strong transcriptional activation domain. After selection by either growing on minimum medium lacking histidine for HIS3 expression or blue color for LacZ expression, the positive clones may then be identified and cDNA isolated. The cDNA may be further characterized to confirm its ability to bind TSE by gel-shift assay or footprinting. One is aware of other methods, such as in vitro expression library screening (Singh et al., 1988) in that they may screen a human mammary gland cDNA expression library with the radioactive labeled TSE as a probe. The positive clones may be further characterized to confirm the TSE binding. In this embodiment there is an inability to detect the TSEBP that requires other auxiliary proteins for DNA binding, and, therefore, one can use the conventional DNA affinity chromatography described previously (Marshak et al., 1996). Similar approaches can be applied to the TSE-binding transcriptional repressor present in cancer cells.

To demonstrate the in vivo binding of the TSEBP to IFIX promoter, one may first generate the specific antibody against TSEBP and test its ability to pull down IFIX protein in an immunoprecipitation assay. If so, one may perform the chromatin immunoprecipitation (CHIP) assay (Weinmann et al., 2001) to show the direct involvement of TSEBP on the IFIX promoter. Briefly, nuclei isolated from the formaldehyde treated normal and cancer cells may be sonicated, followed by immunoprecipitation with TSEBP antibody or the nonspecific antibody as a control. After reverse crosslinks (by high salt, e.g., 200 mM NaCl) and proteinase treatment, the DNA fragments may be PCR synthesized using IFIX promoter-specific primers. In the embodiment wherein TSEBP is a positive factor present in normal cells but not in cancer cells, the TSE-containing IFIX promoter elements may appear in CHIP from normal cells but not in cancer cells. The result may be the opposite if TSEBP is a negative factor present in cancer cells but not in normal cells. The nonspecific control antibodies will not pull down IFIX-specific DNA fragments.

In a specific embodiment of the present invention, the differential IFIX transcriptional activity is caused by mutations on the promoter sequences. In that case, one may make nucleotide substitutions on a promoter sequence to identify the mutation that is responsible for the downregulation of IFIX transcription in cancer cells. Heterologous promoter study and gel-shift assay may be used to further confirm the result of promoter analysis.

In some embodiments, IFIX downregulation is due to genetic alterations, e.g., deletion and rearrangement, of IFIX gene. One may then perform a Southern blot analysis on genomic DNA isolated from the normal and cancer cells and compare the diagnostic patterns specific to certain (about) six nucleotide cutter when hybridized with IFIX probe. Unless there are subtle mutations, the gross DNA alterations should be detected.

### Example 16

### Determination of the Role of P21^{CIP1} in IFIX-Mediated Tumor Suppression

IFIX-mediated tumor suppression activity is associated with the upregulation of p21^{CIP1}, as shown herein. Studies may be performed to establish the significance of p21^{CIP1} in the IFIX-mediated tumor suppression by characterizing the effect of IFIX on p21^{CIP1}-null (or knockdown) cells. Furthermore, IFIX expression upregulates p21^{CIP1} protein, mRNA, and the promoter activity, and in some embodiments p21^{CIP1} promoter is the transcriptional target of IFIX. Studies may be performed to elucidate the mechanism by which IFIX activates p21^{CIP1} promoter.

In the embodiment wherein p21^{CIP1} upregulation is essential for IFIX-mediated anti-tumor activity, one would expect that the IFIX effect will be attenuated on cells that have either no p21^{CIP1} and/or that p21^{CIP1} expression is significantly reduced. Two exemplary approaches may be used to study this: first, an appropriate cell line is obtained, such as the exemplary colon cancer cell line HCT116 and its p21^{CIP1}-null (p21^{-/-}) variant generated by homologous recombination (Waldman et al., 1995). Since these cells harbors both neomycin and hygromycin resistant genes during the generation of the p21^{CIP1}-null cells, it would be difficult to make IFIX stable cell lines from them without using a different selection marker. To circumvent this barrier, one may perform a transient transfection assay in which CMV-IFIX (or the empty vector control) and CMV-GFP at 10:1 ratio will be cotransfected into HCT116 and HCT116 (p21^{-/-}) cells. The GFP-positive cells may be collected by FACS (fluorescence activated cell sorting) analysis and then subjected to MTT and ³H-thymidine incorporation assays. In one embodiment, the growth and proliferation index of HCT116 would be reduced in CMV-IFIX transfected cells, but not in HCT116 (p21^{-/-}) cells. The empty vector control transfection would have no effect on either cell lines.

In the second approach, as described elsewherein herein, one may use both antisense and RNAi techniques to generate the p21^{CIP1}-knockdown (p21-kd) cell lines from breast cancer cell line such as MCF-7 that has detectable level of p21^{CIP1} mRNA and protein. Once the p21-kd cell lines are characterized by both western and northern blot analysis to confirm the downregulation of p21^{CIP1} expression in these cell lines, one may subject them to the transient transfection experiments described above to demonstrate the essential role of p21^{CIP1} upregulation in IFIX-mediated growth inhibition. Furthermore, one may generate IFIX stable cell lines from these p21-kd cells using a different selection marker. The IFIX stable p21-kd cells (IFIX-p21-kd) may be characterized to confirm that IFIX expression is no longer to able to upregulate p21^{CIP1} by western and northern blot analysis. Since the inventors have shown that MCF-7 IFIX stable cell lines (MCF-X-1 and MCF-X-2) had reduced growth rates in anchorage-dependent and -independent manner, in some embodiments the growth inhibitory effect may be impeded in IFIX-p21-kd cells as compared with MCF-X-1 and MCF-X-2. If so, one may perform a tumorigenicity assay by inoculating IFIX-p21-kd and MCF-X-1 (or MCF-X-2) into mfps on either side of the hormone (estrogen pellet)-supplemented female nude mice. The tumor size may be monitored and, in a specific embodiment, there is an increase in tumorigenicity of IFIX-p21-kd cells as compared with MCF-X-1 (or MCF-X-2) cells. The parental MCF-7 tumors will serve as a positive control.

IFIX expression is associated with upregulation of p21^{CIP1} protein and mRNA level, and IFIX could activate p21^{CIP1} promoter activity. These results indicate that p21^{CIP1} promoter may be the transcriptional target of IFIX. In this embodiment, one can perform promoter analysis to identify the cis-acting element through which IFIX activates p21^{CIP1} promoter activity. One may perform the p21^{CIP1} promoter reporter assay in multiple cancer cell lines to select the cell line that gives the best IFIX-mediated activation for further promoter analysis. Once the cell line is identified, one may co-transfect a panel of p21^{CIP1} promoter deletion mutants with CMV-IFIX (or the empty vector) to identify the cis-acting element (XRE, IFIX Responsive Element) responsible for the activation of p21^{CIP1} promoter activity. Again, one may subclone XRE into a heterologous promoter, e.g., pGL2-promoter, as described elsewhere herein, to study whether XRE is able to activate transcription in response to IFIX. Mutagenesis study may be performed to identify the mutations on XRE (mXRE) that would abolish IFIX-mediated transactivation.

Once XRE is identified, the one may test to determine if IFIX is the trans-acting factor that transactivates p21^{CIP1} promoter activity through XRE. One may perform gel-shift assay using radiolabeled XRE as a probe and incubate with nuclear extract isolated from either IFIX stable cell lines, e.g., 468-X-2 and MCF-X-2, or the control cells, 468 and MCF-7. The specific protein/XRE complex may be identified by cold XRE and mXRE competition. In one embodiment, the specific protein/XRE complex is present in IFIX stable cells but not in the control cells. Antibody against IFIX may be added to the binding reaction prior to gel shift assay to examine whether IFIX is present in the specific protein/XRE complex. If not, it indicates that IFIX may activate p21^{CIP1} promoter activity via an indirect mechanism. If IFIX is present in the protein/XRE complex, one may perform studies to test if IFIX directly binds to DNA or indirectly through a protein-protein interaction. One of the ways is to use the in vitro synthesized IFIX protein using a TNT in vitro synthesis kit (Ambion, Inc.; Austin, Tex.) and incubate with the radiolabeled XRE in a gel shift assay. If the protein/XRE complex can be specifically competed by cold XRE but not cold mXRE; and IFIX antibody can supershift this complex, it indicates that IFIX is able to interact with XRE directly.

To demonstrate the in vivo binding of the IFIX to XRE, one may perform a CHIP assay using IFIX specific antibody to show the direct involvement of IFIX on the p21^{CIP1} promoter. One may see that, if IFIX is in the protein/XRE complex, the XRE-containing p21^{CIP1} promoter elements will appear in CHIP from IFIX stable cells but not in the control cells.

Given that IFIX is a nuclear protein and its family members, e.g., IFI16, MNDA, p202, and p204 (Johnstone and Trapani, 1999), have been associated with transcriptional regulation, in some embodiments p21^{CIP1} is not the only transcriptional target for IFIX. One may perform DNA array using IFIX stable cell lines to identify the specific IFIX-regulated genes. Yeast two-hybrid system may also be performed to identify the IFIX-interacting partners. These results will indicate in which signal pathways IFIX is involved and how IFIX regulates the expression of genes in these pathways.

### Example 17

### Determination of the IFIX-Mediated Tumor Suppressor Activity in Pre-Clinical Breast Cancer Gene Therapy Setting

Given that cancer, such as the exemplary breast cancer, is a metastatic disease, systemic treatment is important to achieve therapeutic efficacy and improved survival. Therefore, one may determine the efficacy of IFIX-based gene therapy treatment by systemic delivery. Studies may be performed to demonstrate the IFIX effect on the primary breast tumor tissues and to examine the efficacy of systemic treatment using liposome or adenoviral vector as a gene delivery system.

In one embodiment of the present invention, IFIX effect on the primary breast tumors is beneficial. To this end, the IFIX-mediated growth effect as well as any apoptosis effect on the primary breast tumor tissues may be studied. Briefly, tumor tissues may be enzymatically dissociated in RPMI-1640 medium containing 0.1% collagenase IV, 0.01% hyaluronidase V, 0.002% DNase I, and 1% (v/v) antibiotic-antimycotic into single cell suspension, washed, and cultured (Toloza et al., 1997). These primary breast tumor cells may survive for a few passages in tissue culture. One may then co-transfect these cells with CMV-IFIX (or the empty vector) and CMV-GFP (10:1). Forty-eight hours post-transfection, one may perform BrdU staining for the GFP-positive cells in the S-phase of cell cycle. One expects to see that the % of BrdU-positive cells transfected with CMV-IFIX would be significantly lower than that of the empty vector transfection. Alternatively, one may co-transfect CMV-IFIX (or the empty vector) and CMV-luc (10:1). Forty-eight hours post-transfection, one may perform luciferase assay, which is proportional to the number of viable cells. In an embodiment of the present invention, the luc activity would be significantly lower in CMV-IFIX transfected cells than that in the empty vector transfected cells.

Since breast cancer is a metastatic disease, it is critical to develop a systemic delivery system for IFIX treatment. The present inventors have demonstrated therapeutic efficacy by intra-tumor treatment of CMV-IFIX/SN2 liposome complex in an orthotopic breast cancer xenograft model. Thus, one may determine if IFIX/SN2 treatment would yield therapeutic efficacy using a systemic treatment protocol. Due to the nature of systemic treatment, each mouse may be inoculated with one orthotopic tumor in the mfp. Two weeks post-inoculation of MDA-MB-468 cells, the tumor-bearing mice may be divided into three treatment groups (10 tumors/10 mice/treatment): (i) PBS; (ii) pCMV-Tag2/SN2; and (iii) CMV-IFIX/SN2. SN2 (52 μg) with or without DNA (20 μg) in 200 μl PBS will be given weekly by tail-vein injection. As described herein, the initial treatment frequency may be twice per week for 5 weeks and once per week thereafter. This treatment frequency has been shown effective (Ding et al., 2002). However, the optimal treatment frequency and DNA dose may be determined if detrimental effect on the animals or ineffectiveness was seen using the current protocol. Both tumor volume and survival rate can be measured. MCF-7 xenograft models may be likewise tested if IFIX show promising results in vitro and in vivo. In a specific embodiment, there is a reduction of tumor volume in the CMV-IFIX/SN2-treated mice as compared with the control groups. Immunohistochemical staining and western blot may be used to analyze the expression of IFIX. Since p202, a HIN-200 protein, expression is associated with suppression of metastasis and angiogenesis (Wen et al., 2001) as well as increased apoptosis (Ding et al., 2002), it is likely that IFIX may also have similar effects. One may, therefore, perform immunostaining to study the expression of CD31, VEGF, bFGF, IL-8 and CD34 for the possible anti-angiogenic effect. To examine the possible IFIX-induced apoptosis on the CMV-IFIX/SN2-treated tumors, one may perform TUNEL (TdT (terminal deoxynucleotidy transferase)-mediated dUTP nick end labeling) assay that stains the ends of DNA fragments caused by apoptosis.

Ad-IFIX may be constructed according to the protocol described previously (He et al,. 1998). Briefly, IFIX cDNA may be subcloned into an adenovirus vector (pAd-TRACK-CMV) that carries a CMV promoter-driven green fluorescence protein (GFP). A separate CMV promoter directs IFIX cDNA. pAd-TRACK-CMV (harboring both CMV-GFP and CMV-IFIX) and pAd-EASY1 (containing adenovirus backbone) may be used to transform E. coli (BJ5183) to generate the Ad-IFIX vector by homologous recombination. The recombinant Ad-IFIX vector may be used to transfect 293-CrmA cells to make Ad-IFIX virus. One may verify IFIX protein expression on the Ad-IFIX infected cells by western blot analysis. The control virus, an adenoviral vector expressing luciferase gene and GFP (Ad-luc), has been made (Ding et al,. 2002). The expression of GFP gene enabled the inventors to monitor the infection efficiency by direct observation using a fluorescence microscope.

In specific embodiments, one may infect breast cancer cell lines, e.g. MDA-MB-468 and MCF-7, with Ad-IFIX or Ad-luc, followed by the growth assays such as MMT and ³H-thymidine incorporation. One may find that, like the IFIX stable cell lines, Ad-IFIX infection will lead to growth inhibition. In one embodiment, Ad-IFIX infection causes apoptosis, and this may be tested by performing apoptosis assays, such as PARP cleavage, flow cytometry (sub-G1 cells), and DNA fragmentation, as described previously (Ding et al., 2002).

To determine the efficacy of Ad-IFIX treatment, the following is performed. Briefly, MDA-MB-468 cells (1×10⁶ cells/100 μl) may be implanted into mfps. Treatments may begin when tumor size reaches ˜0.5 cm in diameter, which usually takes about 2 weeks. Either Ad-IFIX or Ad-luc (5×10⁸ pfu) may be i.v. injected via tail vein into the tumor-bearing mice (the tumor size should be comparable among tumor-bearing mice at the time of treatment). Treatments may be administered twice per week for five weeks and once a week thereafter. Tumor volume may be monitored weekly. Mice may be sacrificed when tumor size reaches about 1.5-cm in diameter according to the Institutional Guideline. Upon autopsy, mice may be examined for metastasis, especially in lungs, and breast tumors may be excised for the immunohistochemical and pathological studies. In particular, TUNEL assay may be used to detect apoptotic cells, and CD31 staining may be used to detect the presence of new blood vessels in tumors. Immunohistochemical assays may be used to detect the expression of IFIX, IL-8, MMP-9, VEGF, bFGF, and CD34 in tumor samples.

### Example 18

### IFIX for Treatment of Prostate Cancer

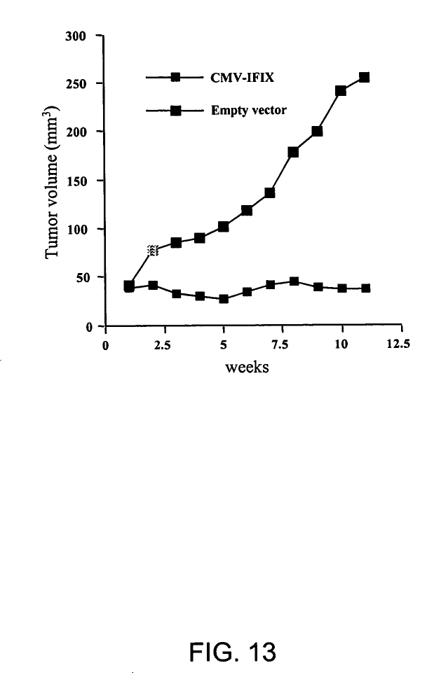

FIG. 13 describes IFIX gene therapy treatment in a human prostate cancer xenograft model. Human prostate cancer cell line (PC-3) (1×10⁶) was implanted subcutaneously on both flanks of male nude mice (2 tumors per mouse, 5 mice per treatment). Treatment began when tumor size reached 5 mm in diameter. Each treatment consists of 15 μg of CMV-IFIX (or empty vector) and 15 μl of SN2 liposome by intratumoal injection at twice a week. Tumor volume did not increase in mice treated with CMV-IFIX, as opposed to the control mice treated with empty vector.

### Example 19

### IFIXα1 Activates P53 by Downregulating HDM2

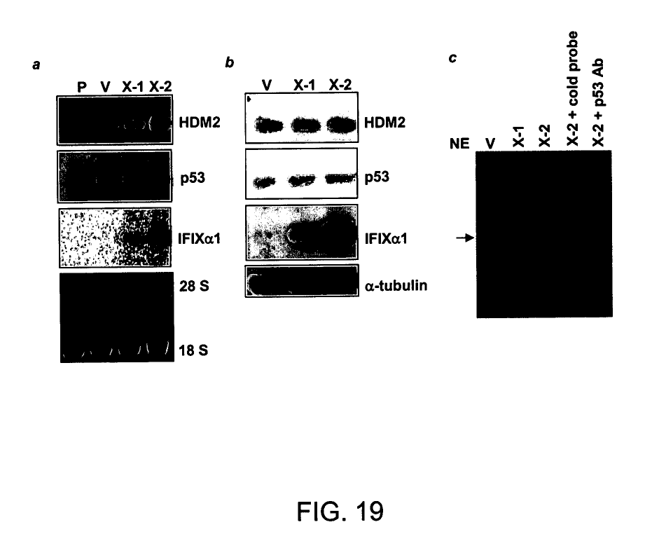

As demonstrated herein, IFIXα1 downregulates HDM2 and thereby activates p53, which indicates at least one potent mechanism for its anti-tumor activity and utility as a cancer therapeutic. In FIG. 19A, HDM2 mRNA levels were increased by IFIXα1. Total RNA (10 μg) isolated from MCF-7 parental (P), vector (V) control, and IFIXα1 stable cell lines (X-1 and X-2) were analyzed by northern blot using an HDM2, p53, or IFIXα1 probe as indicated. The 18S and 28S rRNA bands serve as loading control. In FIG. 19B, there are increased p53 protein levels by IFIXα1. Total cell lysates were analyzed by western blot using antibodies against HDM2, p53, IFIXα1, and α-tubulin (as a loading control). In FIG. 19C, IFIXα1 enhances p53 DNA binding activity. Nuclear extracts (NE) (7.5 μg) isolated from each cell were incubated with ³²P-labeled oligonucleotide containing p53-binding sites prior to electrophoretic mobility shift assay according to manufacturer's instruction (p53 Nushift kit, Geneka Biotechnology, Inc.; Montreal, Quebec). The cold probe (100-fold excess) was used as competitor. Anti-p53 antibody was added to the binding reaction prior to electorphoresis. The arrow indicates the specific p53/DNA complex.

### Example 20

### IFIXα1 Promotes HDM2 Protein Degradation

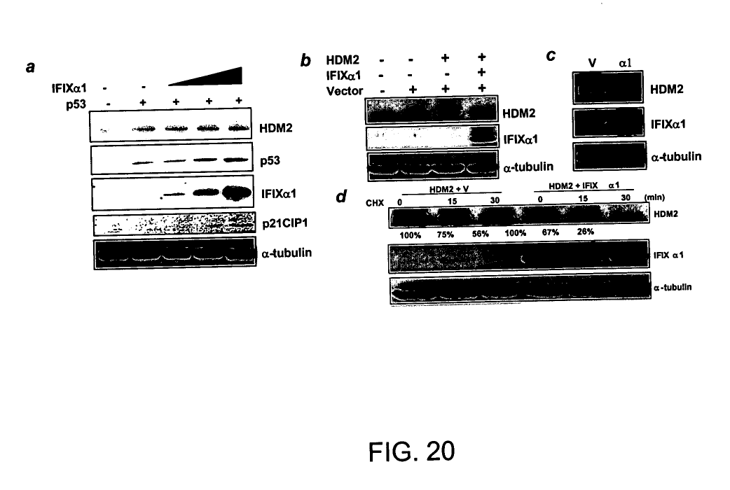

This Example demonstrates that IFIXα1 promotes HDM2 protein degradation. In FIG. 20A, the HDM2 protein levels are not p53 dose-dependent in the presence of IFIXα1. H1299 cells were transfected with increased amount of IFIXα1 (0.5, 1.0, 1.8 μg) and p53 (0.1 μg). Twenty-four h post-transfection, cell lysates were isolated for western blot analysis using antibodies against HDM2, p53, IFIXα1, p21^{CIP1}, and α-tubulin (as a loading control). In FIG. 20B, IFIXα1 reduces HDM2 protein levels in the absence of p53. H1299 cells transfected with with pcDNA3 (Vector) (1.5 μg), HDM2 (0.8 μg)+Vector (0.7 μg), or HDM2 (0.8 μg)+IFIXα1 (0.7 μg). Forty h post-transfection, cell lysate was isolated followed by western blot using anti-HDM2, anti-IFIX, or anti-α-tubulin antibody. In FIG. 20C, IFIXα1 expression reduces the HDM2 levels. 293T cells were transfected with 2 μg of either EGFP vector (V) or EGFP-IFIXα1 (α1) followed by western blot using anti-HDM2, anti-IFIX, or anti-a-tubulin antibody. In FIG. 20D, IFIXα1 reduces the half-life of HDM2 protein. H1299 cells were co-transfected with HDM2 (0.7 μg) and 1.3 μg of either pcDNA3 (V) or IFIXα1. Twenty-four h post-transfection, cells were treated with cyclohexamide (CHX) (100 μg/ml). Cell lysates were isolated at 0, 15, and 30 min after CHX treatment for western blot analysis using antibodies against HDM2, IFIXα1, and α-tubulin (as a loading control). The amount of HDM2 protein at 0 time point was set at 100%. The % of HDM2 protein remained is indicated as determined by using BioRad software.

### Example 21

### IFIXα1 Interacts with HDM2

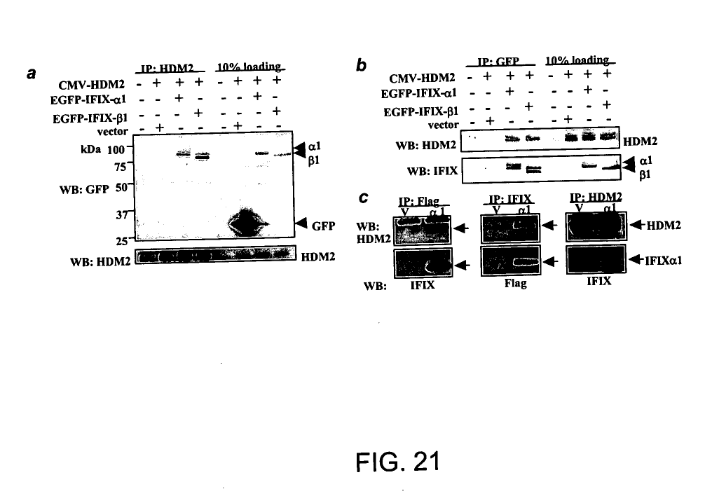

As shown herein, IFIXα1 interacts with HDM2. FIG. 21A shows that HDM2 interacts with EGFP-IFIXα1 and β1. 293T cells were transfected with 2.5 μg CMV-HDM2 and 2.5 μg CMV-vecter (Vector), or 2.5 μg EGFP-IFIXα1 (α1), EGFP-IFIXβ1 (β1). Forty-eight h post-transfection, cell extracts (500 μg) were immunoprecipitated (IP) with a monoclonal anti-HDM2 antibody (Santa Cruz), and western blot (WB) was performed using a polyclonal anti-GFP (Abcam) or anti-MDM2 (Santa Cruz) antibody. In FIG. 21B, there is a reciprocal experiment that used anti-GFP antibody for IP and WB with anti-IFIX or anti-HDM2 antibodies. In FIG. 21C, HDM2 interacts with Flag-tagged IFIXα1. 293T cells were transfected with 5 μg CMV-HDM2 and 5 μg Flag-vector (V) or Flag-IFIXα1 (α1). Forty-eight h post-transfection, cell extracts (500 μg) were IP using anti-Flag (M5, Sigma), anti-IFIX, or anti-HDM2 antibodies and WB using anti-IFIX, anti-Flag, and anti-IFIX antibody, respectively. Although the interaction in the complex between IFIXα1 and HDM2 may be direct, in specific embodiments, the interaction may alternatively be indirect, in some embodiments.

### Example 22

### Testing of Exemplary IFIX as Therapeutic Agents

At least one IFIX as it relates to anti-tumor activity is tested in an animal study, such as cell lines, cell culture, and/or models in addition to or other than those described in the preceding Examples. In general embodiments of the present invention, IFIX is delivered by a vector, such as a liposome, adenoviral vector, or combination thereof, into nude mice models for its anti-tumor activity. Once the anti-tumor activity is demonstrated, potential toxicity is further examined using immunocompetent mice, followed by clinical trials.

In a specific embodiment, the preferential growth inhibitory activity of IFIX is tested in animal. Briefly, cancer cell lines are administered into mammary fat-pad of nude mice to generate a breast xenografted model. Although, as described herein, any cancer cell is within the scope of the present invention irrespective of its genotype or expression levels, (such as, for example, whether it is HER-2/neu-positive or HER-2/neu-negative), in a specific embodiment HER-2/neu overexpressing breast cancer cell lines (such as, for example, SKBR3 and/or MDA-MB361) are utilized, such as for testing. After the tumors reach a particular size, the IFIX and/or wild-type IFIX control is administered into the mouse, such as, for example, intravenously injected in an admixture with an acceptable carrier, such as liposomes. The tumor sizes and survival curve from these treatments are compared and statistically analyzed. In a preferred embodiment, the IFIX is substantially the same as or better in its inhibition of the growth of tumor compared to that of wild-type IFIX.

### Example 23

### Preparation of Additional Forms of IFIX

Based on the data in previous Examples and the teachings elsewhere in the specification, in addition to the knowledge in the art, a skilled artisan would be motivated and capable of generating additional forms of IFIX and, furthermore, able to determine the usefulness in the context of the invention using methodology disclosed herein.

### Example 24

### Testing of Additional IFIX

Once IFIXs other than the exemplary embodiments disclosed herein are created, testing using a cell culture in a relevant cell line(s) is performed, such as described herein. Furthermore, testing of the IFIXs using FACS analysis is performed, such as described herein. Also, testing of the additional IFIXs using ex vivo systems or in vivo systems as described herein may be employed, in specific embodiments.

### Example 25

### Clinical Trials

This example is concerned with the development of human treatment protocols using the IFIX protein, peptide, or polypeptide or a nucleic acid encoding the IFIX protein, peptide, or polypeptides, alone or in combination with other anti-cancer drugs. The IFIX protein, peptide, or polypeptide or a nucleic acid encoding the IFIX protein, peptide, or polypeptides, and anti-cancer drug treatment will be of use in the clinical treatment of various cancers involving, for example, Akt activation in which transformed or cancerous cells play a role. Such treatment will be particularly useful tools in anti-tumor therapy, for example, in treating patients with ovarian, breast, prostate, pancreatic, brain, colon, and lung cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the IFIX protein, peptide, or polypeptide or a nucleic acid encoding the IFIX protein, peptide, or polypeptides, in clinical trials.

Patients with advanced, metastatic breast, epithelial ovarian carcinoma, pancreatic, colon, or other cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the the IFIX protein, peptide, or polypeptide or a nucleic acid encoding the IFIX protein, peptide, or polypeptides, and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (IFIX proteins, peptides or polypeptides or nucleic acids encoding the same) may be assessed and recorded.

In the same procedure, the IFIX protein, peptide, or polypeptide or a nucleic acid encoding the IFIX protein, peptide, or polypeptides, may be administered alone or in combination with the other anti-cancer drug. The administration may be in the pleural/peritoneal cavity, directly into the tumor, or in a systemic manner. The starting dose may be 0.5 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the IFIXprotein, peptide, or polypeptide or a nucleic acid encoding the IFIX protein, peptide, or polypeptides, and the lot of anti-cancer drug exceed 5 EU/kg for any given patient.

The IFIX protein, peptide, or polypeptide or a nucleic acid encoding the IFIX protein, peptide, or polypeptides, and/or the other anti-cancer drug combination, may be administered over a short infusion time or at a steady rate of infusion over a 7 to 21 day period. The IFIX protein, peptide, or polypeptide or a nucleic acid encoding the IFIX protein, peptide, or polypeptides, infusion may be administered alone or in combination with the anti-cancer drug and/or emodin like tyrosine kinase inhibitor. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the IFIX protein, peptide, or polypeptide or a nucleic acid encoding the mutant protein, peptide, or polypeptides, in combination with an anti-cancer drug will be administered to groups of patients until approximately 60% of patients show unacceptable Grade III or IV toxicity in any category. Doses that are 2/3 of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms) and Akt (phosphorylated and non-phosphorylated forms), p185, and so forth.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, ki67 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### Example 26

### Nuclear Localization of IFIX

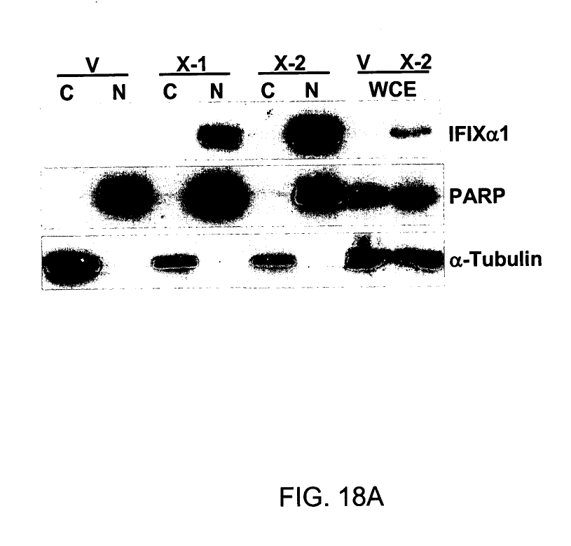

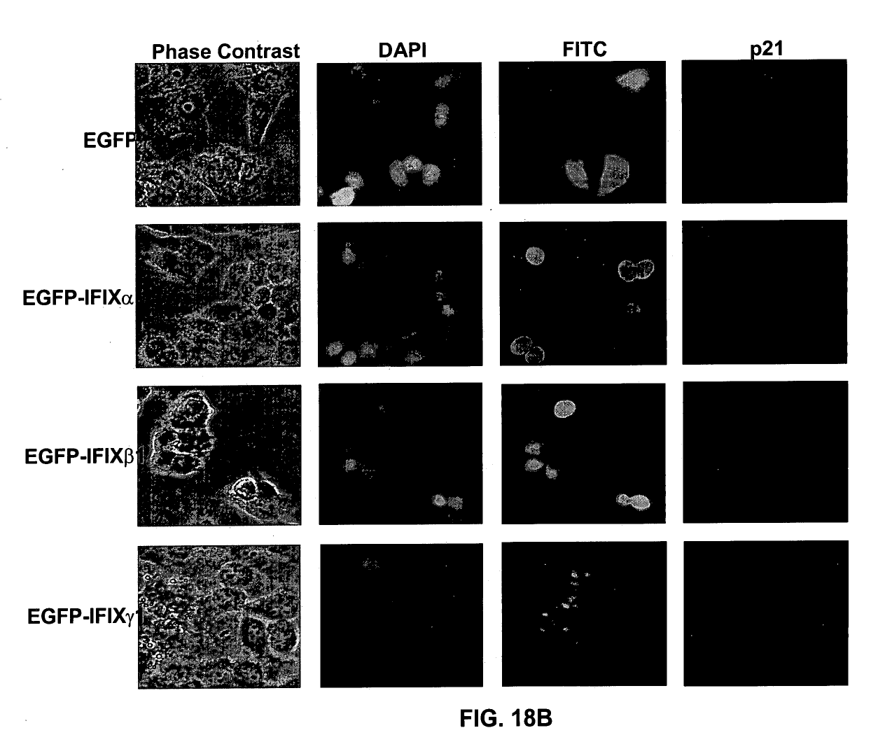

The N-terminal region also contains a putative nuclear localization signal (Dawson & Trapani, 1995), comprising ¹³⁴LGPQKRKK (SEQ ID NO:23), although in some embodiments it comprises ¹³⁶PQKRKK (SEQ ID NO:22) (FIG. 7). To characterize the ability of different IFIX isoforms to induce p21^{CIP1}, the present inventors transiently transfected MCF-7 cells with the plasmids encoding EGFP-tagged IFIXα1, β1, or γ1 fusion protein followed by immunostaining with the p21^{CIP1} specific antibody. As shown in FIG. 18B, the expression of IFIXα1 or β1 mainly coincides with the expression of p21^{CIP1} in the nucleus (64% and 52%, respectively). In contrast, like the empty vector (EGFP) control, the expression of IFIX γ1 has little effect on the expression of p21^{CIP1} (0.95% and 2%, respectively). Together with a unique speckled nuclear pattern, our observations indicate IFIX γ1 may function differently from IFIXα1/β1; and it also suggests that the 200-amino-acid domain may be responsible for the up-regulation of p21^{CIP1}.

Consistent with this indication, the present inventors found that the stably transfected IFIXα1 as well as the EGFP-tagged IFIXα1, β1, and γ1 fusion proteins are localized in the nucleus (FIGS. 18A and 18B). Interestingly, while IFIXα1 and IFIX β1 are primarily localized in the nucleoplasm, IFIX γ1 forms a speckled nuclear pattern (FIG. 18B). This observation indicates that, like most of the HIN-200 proteins, the IFIX proteins are primarily nuclear proteins.

Methods for this example are as follows, although a skilled artisan recognizes that optimized, modified, or analogous methods may also be utilized. MCF-7 cells (1×10⁴ in 0.5 ml) were cultured in a 4-well glass chamber overnight. Cells were then transfected with 1 μg of the plasmid encoding EGFP-tagged IFIXα1, β1, or γ1 fusion protein. The EGFP expression vector serves as a control. Forty-eight hours after transfection, cells were washed with PBS and fixed with 3% paraformadehyde in PBS for 20 min at room temperature followed by PBS wash. The primary p21^{CIP1} monoclonal antibody (Santa Cruz Biotech.) (1:100) was incubated with the cells at 37° C. for 1 hour. Cells were then washed with PBS followed by incubation with the rabbit anti-mouse secondary antibody conjugated with Texas Red (1:200) at 37° C. for 45 min. After incubation, cells were washed briefly with PBS and air-dried followed by incubation with the blue fluorescent dye DAPI (1:100 in 50% Glycerol/PBS). A cover slip was placed on top of the slide for visualization by microscopy.

### Example 27

### Significance of the Results

IFIX is a novel member of human HIN-200 protein family. At least six alternatively spliced variants have been identified, four of which (α1, α2, β1 and β2) possess a type a 200-amino acid repeat. The functional role of the 9 amino acids (VANKIESIP) absent in α2, β2, and γ2 remains unknown. However, like the stable cell lines expressing IFIXα1 (FIG. 3d), cells expressing IFIXα2, β1, or β2 also exhibited reduced tumorigenicity. These observations indicate that in some embodiments neither these 9 amino acids nor the C-terminal domains different between α and β isoforms play a role in tumor suppression. Although it has been suggested that both type a and b of the 200-amino acid repeats are required for growth suppression (Gribaudo et al., 1999), the data provided herein indicate that support the idea HIN-200 proteins containing only a type a repeat, like IFIX, are able to inhibit cell growth (Choubey et al., 2000). One may perform studies to determine if the 200-amino acid repeat is required for growth suppression by expressing IFIXγ1 or γ2 in the cells, because these isoforms lack the 200-amino acid repeat. As shown herein, IFIX was down-regulated in 26 out of 50 breast cancers in a commercial cDNA expression array (FIG. 9) as well as in 5 breast cancers collected from patients (FIG. 2a). Consistent with the breast tissue data (FIGS. 8 and 2a), 7 out of 9 breast cancer cell lines examined in this study have no detectable IFIX, while IFIX expression is readily detectable in the non-transformed breast cell lines (FIG. 2b). These results indicate that IFIX expression is reduced during tumorigenesis. IFIX-specific antibodies may be generated to detect IFIX proteins by western blot and immunostaining. Once available, a more systematic analysis of IFIX expression on breast tumor tissues may be performed. The expression of HIN-200 was originally identified in hematopoietic cells and was thought to be restricted to this cell type (Lengyel et al., 1995; Dawson and Trapani, 1996). However, recent reports have shown that IFI16 is expressed in epithelial cells in addition to lymphoid cells (Gariglio et al,. 2002; Wei et al., 2003). The finding that IFIX expresses in normal breast tissues (FIG. 2a) and non-transformed breast epithelial cell lines (FIG. 2b) indicates that HIN-200 expression is not restricted to hematopoietic cells. Taken together, these observations indicate IFIX plays a role in maintaining the normal growth of epithelial cells and the down-regulation of IFIX expression contributes to the uncontrolled cell growth and leads to tumorigenesis.

Based on the ability of IFIX to suppress growth, transformation, and tumorigenicity of breast cancer cells (FIG. 3), an IFIX-based gene therapy was tested to determine if it would yield efficacy in an orthotopic breast cancer xenograft model. Direct injection of IFIX, together with the liposome SN2, into tumors yielded a significant anti-tumor activity as compared to the empty vector control (FIG. 4). Since breast cancer is a metastatic disease, this observation illustrates the therapeutic efficacy of IFIX in systemic treatments delivered by either SN2 liposome (Zou et al., 2002) or viral vectors (Ding et al., 2002). IFN has been shown to increase the expression of p21^{CIP1}, which is critical for IFN to suppress the anchorage-independent growth of breast cancer cells (Gooch et al., 2000). The results show expression of IFIX reduces the growth of breast cancer cells in soft agar (FIG. 3c) and increases the expression of the CKI p21_{CIP1}, but not other CKIs such as p27_{KIP1}, p57_{KIP2}, and p16_{INK4a}. The result that IFIX is able to up-regulate p21^{CIP1} in MDA-MB-468 cells, which express only mutant p53, indicates that the up-regulation of p21^{CIP1} by IFIX is independent of p53. This observation is consistent with a previous finding that p202a overexpression resulted in a p53-independent up-regulation of p21^{CIP1} (Gutterman and Choubey, 1999). As expected, the up-regulation of p21^{CIP1} leads to hypo-phosphorylation of pRb in IFIX-expressing MCF-7 cells. However, since MDA-MB-468 cells lack pRb (28, 29), the inhibition of E2F/pRb pathway by p21^{CIP1} cannot account for the mechanism for IFIX-mediated growth inhibition of MDA-MB-468 cells. The finding that the kinase activity of p34^{CDC2} is reduced in IFIX-expressing MDA-MB-468 cells (FIG. 5c) indicates that IFIX suppresses the growth of MDA-MB-468 cells through the inhibition of p34^{CDC2} kinase activity at the G2/M phase of cell cycle by p21^{CIP1}. IFIX, a newly identified HIN-200 gene, is down-regulated in breast cancer. The data presented indicate IFIX expression is associated with tumor suppressor activity and p21^{CIP1} up-regulation in breast cancer. Moreover, efficacy of an IFIX-based gene therapy is demonstrated, showing the utility of using IFIX as a therapeutic agent in breast cancer treatment.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents

U.S. Pat. No. 4,683,202
U.S. Pat. No. 4,797,368
U.S. Pat. No. 4,879,236
U.S. Pat. No. 5,139,941
U.S. Pat. No. 5,220,007
U.S. Pat. No. 5,284,760
U.S. Pat. No. 5,354,670
U.S. Pat. No. 5,366,878
U.S. Pat. No. 5,389,514
U.S. Pat. No. 5,635,377
U.S. Pat. No. 5,641,484
U.S. Pat. No. 5,670,488
U.S. Pat. No. 5,789,166
U.S. Pat. No. 5,798,208
U.S. Pat. No. 5,830,650
U.S. Pat. No. 5,871,986
U.S. Pat. No. 5,925,565
U.S. Pat. No. 5,928,906
U.S. Pat. No. 5,935,819

### Publications

Aldaz, C. M., Liao, Q. Y., LaBate, M. & Johnston, D. A. Medroxyprogesterone acetate accelerates the development and increases the incidence of mouse mammary tumors induced by dimethylbenzanthracene. Carcinogenesis 17, 2069-72 (1996).
Brenning, G., Ahre, A. & Nilsson, K. (1985) Scand J Haematol 35, 543-549.
Cardiff, R. D. & Muller, W. J. Transgenic mouse models of mammary tumorigenesis. Cancer Surveys 16, 97-113 (1993).
Carlson, C. A. & Ethier, S. P. (2000) Radiat Res 154, 590-599.
Chiba, H., Clifford, J., Metzger, D. & Chambon, P. Distinct retinoid X receptor-retinoic acid receptor heterodimers are differentially involved in the control of expression of retinoid target genes in F9 embryonal carcinoma cells. Molecular & Cellular Biology 17, 3013-20 (1997).
Choubey, D. (2000) J Biol Regul Homeost Agents 14, 187-192.
Choubey, D., Walter, S., Geng, Y. & Xin, H. (2000) FEBS Lett 474, 38-42.
Dawson, M. J. & Trapani, J. A. (1996) J Leuko Biol 60, 310-316.
Derman, E. et al. Transcriptional control in the production of liver-specific mRNAs. Cell 23, 731-9 (1981).
Ding, Y. et al. Pro-apoptotic and anti-tumo activities of adenovirus-mediated p202 gene transfer. Clin Cancer Res in press (2002).
Ding, Y., Wen, Y., Spohn, B., Wang, L., Xia, W., Kwong, K. Y., Shao, R., Li, Z., Hortobagyi, G. N., Hung, M.-C. & Yan, D.-H. (2002) Clin Cancer Res 8, 3290-3297.
G., Voglino, G., Navino, L. & Landolfo, S. (2002) J Interferon Cytokine Res 22, 815-821.
Gariglio, M., Azzimonti, B., Pagano, M., Palestro, G., De Andrea, M., Valente,
Gooch, J. L., Herrera, R. E. & Yee, D. (2000) Cell Growth Differ 11, 335-342.
Gribaudo, G., Riera, L., De Andrea, M. & Landolfo, S. (1999) FEBS Lett 456, 31-36.
Gutterman, J. U. & Choubey, D. (1999) Cell Growth Differ 10, 93-100.
Guy, C. T. et al. Expression of the neu protooncogene in the mammary epithelium of transgenic mice induces metastatic disease. Proc Natl Acad Sci USA 89, 10578-82 (1992).
Hannon, G. J. RNA interference. Nature 418, 244-51 (2002).
He, T. C. et al. A simplified system for generating recombinant adenoviruses. Proc Natl Acad Sci USA 95, 2509-14 (1998).
Johnstone, R. W. & Trapani, J. A. (1999) Mol Cell Biol 19, 5833-5838.
Johnstone, R. W. & Trapani, J. A. Transcription and growth regulatory functions of the HIN-200 family of proteins. Mol Cell Biol 19, 5833-8 (1999).
Kimchi, A., Resnitzky, D., Ber, R. & Gat, G. (1988) Mol Cell Biol 8, 2828-2836.
Landolfo, S., Gariglio, M., Gribaudo, G. & Lembo, D. (1998) Biochimie 80, 721-728.
Lengyel, P. (1993) Proc. Natl. Acad. Sci. USA 90, 5893-5895.
Lengyel, P., Choubey, D., Li, S.-J., & Datta, B. (1995) Semi Virol 6, 203-213 14.
Lengyel, P., Choubey, D., Li, S.-J., and Datta, B. The interferon-activatable gene 200 cluster: from structure toward function. Seminar in Virology 6, 203-213 (1995).
MacLachlan, T. K., Sang, N. & Giordano, A. (1995) Crit Rev Eukaryo Gene 15 Express 5, 127-156.
Marshak, S., Totary, H., Cerasi, E. & Melloul, D. Purification of the beta-cell glucose-sensitive factor that transactivates the insulin gene differentially in normal and transformed islet cells. Proceedings of the National Academy of Sciences of the United States of America 93, 15057-62 (1996).
Matsuoka, M., Tani, K. & Asano, S. (1998) Oncogene 16, 2075-2086.
Muller, W. J., Sinn, E., Pattengale, P. K., Wallace, R. & Leder, P. Single-step induction of mammary adenocaricinoma in transgenic mice bearing the activated c-neu oncogene. Cell 54, 105-15 (1988).
Naldini, A., Carney, D. H., Pucci, A. & Carraro, F. (2002) J Cell Physiol 191, 290-297.
Niculescu, A. B., 3rd, Chen, X., Smeets, M., Hengst, L., Prives, C. & Reed, S. I. (1998) Mol Cell Biol 18, 629-643.
Paddison, P. J., Caudy, A. A. & Hannon, G. J. Stable suppression of gene expression by RNAi in mammalian cells. Proceedings of the National Academy of Sciences of the United States of America 99, 1443-8 (2002).
Paddison, P. J., Caudy, A. A., Bernstein, E., Hannon, G. J. & Conklin, D. S. Short hairpin RNAs (shRNAs) induce sequence-specific silencing in mammalian cells. Genes & Development 16, 948-58 (2002).
Pestka, S., Langer, J. A., Zoon, K. C., & Samuel, C. E. (1987) Annu. Rev. Biochem. 56, 727-777.
Shao, R., Karunagaran, D., Zhou, B. P., Li, K., Lo, S.-S., Deng, J., Chiao, P., & Hung, M.-C. (1997) J. Bio. Chem. 272, 32739-32742.
Singer, B. & Kusmierek, J. T. Chemical mutagenesis. Annu Rev Biochem 51, 655-93 (1982).
Singh, H., LeBowitz, J. H., Baldwin, A. S., Jr. & Sharp, P. A. Molecular cloning of an enhancer binding protein: isolation by screening of an expression library with a recognition site DNA. Cell 52, 415-23 (1988).
Slamon, D. J. et al. Human breast cancer: correlation of relapse and survival with amplification of the HER-2/neu oncogene. Science 235, 177-82 (1987).
Sui, G. et al. A DNA vector-based RNAi technology to suppress gene expression in mammalian cells. Proceedings of the National Academy of Sciences of the United States of America 99, 5515-20 (2002).
Toloza, E. M. et al. Transduction of murine and human tumors using recombinant adenovirus vectors. Annals of Surgical Oncology 4, 70-9 (1997).
Waldman, T., Kinzler, K. W. & Vogelstein, B. p21 is necessary for the p53-mediated G1 arrest in human cancer cells. Cancer Research 55, 5187-90 (1995).
Wei, W., Clarke, C. J. P., Somers, G. R., Cresswell, K. S., Loveland, K. A., Trapani, J. A. & Johnstone, R. W. (2003) Histochem. Cell. Biol. 119, 45-54.
Weinmann, A. S., Bartley, S. M., Zhang, T., Zhang, M. Q. & Farnham, P. J. Use of chromatin immunoprecipitation to clone novel E2F target promoters. Molecular & Cellular Biology 21, 6820-32 (2001).
Wen, Y. et al. p202, an interferon-inducible protein, mediates multiple anti-tumor activities in human pancreatic cancer xenograft models. Cancer Res 61, 7142-7147 (2001).
Wen, Y. et al. Tumor suppression and sensitization to tumor necrosis factor alpha-induced apoptosis by an interferon-inducible protein, p202, in breast cancer cells. Cancer Res 60, 42-6 (2000).
Wen, Y., Yan, D. H., Spohn, B., Deng, J., Lin, S. Y. & Hung, M. C. (2000) Cancer Res 60, 42-46.
Wen, Y., Yan, D.-H., Wang, B., Spohn, B., Ding, Y., Shao, R., Zhou, Y., Xie, K.& Hung, M.-C. (2001) Cancer Res 61, 7142-7147.
Xia, W. et al. Combination of EGFR, HER-2/neu, and HER-3 is a stronger predictor for the outcome of oral squamous cell carcinoma than any individual family members. Clin Cancer Res 5, 4164-74. (1999).
Yan, D.-H., Wen, Y., Spohn, B., Choubey, D., Gutterman, J. U., & Hung, M.-C. (1999) Oncogene 18, 807-811.
Yin, F., Giuliano, A. E., Law, R. E. & Van Herle, A. J. (2001) Anticancer Res 21, 413-420.
Yu, D., Jing, T., Liu, B., Yao, J., Tan, M., McDonnell, T. J. & Hung, M. C. (1998) Mol Cell 2, 581-591.
Zhou, Y., Wang, S., Yue, B. G., Gobl, A. & Oberg, K. (2002) Cancer Invest 20, 348-356.
Zou, Y., Peng, H., Zhou, B., Wen, Y., Wang, S. C., Tsai, E. M. & Hung, M. C. (2002) Cancer Res 62, 8-12.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An isolated polynucleotide comprising SEQ ID NO:1, SEQ ID NO:2, SEQ ID NO:3, or SEQ ID NO:4.

2. The polynucleotide of claim 1, wherein the polynucleotide encodes a polypeptide comprising tumor suppressor activity, anti-cell proliferative activity, pro-apoptotic activity, cell cycle arrest-inducing activity or a combination of any two or more of these.

3. An isolated polypeptide comprising SEQ ID NO:5, SEQ ID NO:6, SEQ ID NO:7, or SEQ ID NO:8.

4. The polypeptide of claim 3, wherein the polypeptide comprises tumor suppressor activity, anti-cell proliferative activity, pro-apoptotic activity, cell cycle arrest-inducing activity, or a combination of any two or more of these.

5. The polypeptide of claim 3, further defined as a composition in a pharmacologically acceptable excipient in which the polypeptide is dispersed.

6. The polypeptide of claim 3, further defined as comprised in a pharmacologically acceptable excipient.

7. The polypeptide of claim 3, further defined as being comprised in a suitable container in a kit.

8. The polypeptide of claim 3, further defined as being comprised in a cell.

9. The polypeptide of claim 8, wherein said polypeptide is localized in the nucleus of the cell.

10. A method comprising administering to a cell an IFIX polypeptide.

11. The method of claim 10, wherein the polypeptide further comprises a protein transduction domain.

12. The method of claim 10, further defined as administering the IFIX polypeptide to the nucleus of said cell.

13. The method of claim 10, wherein the cell is comprised in an animal.

14. The method of claim 13, wherein the animal is a human.

15. The method of claim 14, wherein the human has a proliferative cell disorder.

16. The method of claim 15, wherein the proliferative cell disorder is cancer.

17. The method of claim 16, wherein the cancer is breast cancer, prostate cancer, ovarian cancer, sarcoma, lung cancer, brain cancer, pancreatic cancer, liver cancer, bladder cancer, gastrointestinal cancer, leukemia, lymphoma, or myeloma.

18. The method of claim 17, wherein the cancer is breast cancer.

19. The method of claim 18, wherein the cancer is estrogen receptor positive, is estrogen receptor independent, is EGF receptor independent, is EGF receptor overexpressing, is Her2/neu-overexpressing, is not Her-2/neu-overexpressing, is Akt overexpressing, is androgen independent, is p53-independent, is p53-dependent, or a combination thereof.

20. The method of claim 19, wherein the cancer is estrogen receptor independent and is EGF receptor independent.

21. The method of claim 15, wherein the proliferative cell disorder is restenosis.

22. The method of claim 10, wherein the polypeptide is comprised in pharmacologically acceptable excipient.

23. The method of claim 22, wherein the polypeptide is complexed with a lipid.

24. The method of claim 10, wherein administering to the cell an IFIX polypeptide comprises administering to the individual a nucleic acid encoding an IFIX polypeptide.

25. The method of claim 24, wherein the nucleic acid is comprised in a plasmid, a retroviral vector, an adenoviral vector, an adeno-associated viral vector, or a liposome.

26. The method of claim 24, wherein the nucleic acid is dispersed in a pharmacologically acceptable excipient.

27. The method of claim 10, further defined as a method of preventing growth of a cell in an individual.

28. A method of inhibiting cell proliferation comprising contacting a cell with an IFIX polypeptide in an amount effective to inhibit the cell proliferation.

29. The method of claim 28, wherein the IFIX composition comprises an IFIX polypeptide further defined as having tumor suppressor activity, anti-cell proliferative activity, pro-apoptotic activity, cell cycle arrest-inducing activity or a combination of any two or more of these.

30. The method of claim 28, wherein the contacting step is further defined as delivering the IFIX polypeptide to the nucleus of said cell.

31. A method of treating a proliferative cell disorder in an individual comprising the step of administering to the individual an IFIX composition.

32. The method of claim 31, wherein the IFIX composition comprises an IFIX polypeptide further defined as having tumor suppressor activity, anti-cell proliferative activity, pro-apoptotic activity, cell cycle arrest-inducing activity, or a combination of any two or more of these.

33. A method of treating cancer in an individual having the cancer, comprising contacting at least one cancer cell of the individual with a therapeutically effective amount of a polynucleotide encoding an IFIX polypeptide, wherein the polynucleotide is comprised in an liposome.

34. An isolated polynucleotide encoding a polypeptide of SEQ ID NO:5, SEQ ID NO:6, SEQ ID NO:7, or SEQ ID NO:8.

35. The polynucleotide of claim 34, wherein said polynucleotide is further defined as comprising a region of SEQ ID NO: 1, SEQ ID NO:2, SEQ ID NO:3, or SEQ ID NO:4.

36. The polynucleotide of claim 34, wherein the polypeptide comprises tumor suppressor activity, anti-cell proliferative activity, pro-apoptotic activity, cell cycle arrest-inducing activity, or a combination of any two or more of these.

37. The polynucleotide of claim 34, further defined as a composition in a pharmacologically acceptable excipient in which the polypeptide is dispersed.

38. The polynucleotide of claim 37, further defined as being comprised in a suitable container in a kit.


SEQUENCE LISTING (23 sequences)

SEQ ID NO: 1 - DNA, 1749 bp, Homo sapiens
aggaagccagcactagtcaagtgactctaagaagggccattttcccttgttgtctttgaaaatacttcattttcttagcatttcaggagattataacatcctgtatttcagtttctgagagctttactgactgatttccctattcaaaacaatcctcatttcctacatttctgaagatctcaagatctggactactgttgaagaaattcccagtaaggctcacttatatctttagagatggcaaataactacaagaaaattgttctactgaaaggattagaggtcatcaatgattatcattttagaattgttaagtccttactgagtaacgatttaaaacttaatccaaaaatgaaagaagagtatgacaaaattcagattgctgacttgatggaggaaaagttcccaggtgatgccggtttgggcaaactaatagaattcttcaaagaaataccaacactgggagaccttgctgaaactcttaaaagagaaaagttaaaagttgcaaataaaattgaatccattccagtcaaaggaataatcccatctaaaaagacgaaacagaaagaagtgtatcctgctacacctgcatgcaccccaagcaaccgtctcacagctaaaggagcagaggagactcttggacctcagaaaagaaaaaaaccatctgaagaagagactggaaccaaaaggagtaagatgtccaaagagcagactcggccttcctgctctgcaggagccagcacgtccacagccatgggccgttccccacctccccagacctcatcatcagctccacccaacacttcctcaactgagagcctaaaaccattggccaaccgtcacgcaactgccagtaaaaatattttccgagaagacccaataatcgcgatggtactaaatgcaacaaaagtatttaaatatgaatcctcagaaaatgagcaaagaagaatgtttcatgctacagtggctacgcagacacagttctttcatgtgaaggttttaaacatcaacttgaagaggaaattcattaaaaagagaatcatcattatatcaaattattccaaacgtaatagtctcctagaggtgaatgaagcctcttctgtatctgaagctggtcctgaccaaacgtttgaggttccaaaggacatcatcagaagagcaaagaaaattccgaagatcaatattcttcacaaacaaacttcaggatatattgtatatggattatttatgctacatacgaaaattgtaaataggaagacgacaatctatgaaattcaggataaaacaggaagtatggctgtagtaggaaaaggagaatgccacaatatcccctgtgaaaaaggagataagcttcgactcttctgctttcgactgagaaagagggaaaatatgtcaaaactgatgtcagaaatgcatagtttcatccagatacagaaaaatacaaaccagagaagccatgactccaggagcatggcactaccccaggaacagagtcagcatccaaaaccttcagaggccagcacaaccctacctgaaagccatctcaagactcctcagatgccaccaacaaccccatccagcagttccttcaccaagaaggatgaaacccacccaggagcacagtcatcgcctgcaaactttagaatcacctcaccaactgtggcccctcctctttcttctgacacttccaccaaccgccatccagcagttccttaaataaggtcaccaaggacaaggatatcaaataac

SEQ ID NO: 2 - DNA, 1722 bp, Homo sapiens
aggaagccagcactagtcaagtgactctaagaagggccattttcccttgttgtctttgaaaatacttcattttcttagcatttcaggagattataacatcctgtatttcagtttctgagagctttactgactgatttccctattcaaaacaatcctcatttcctacatttctgaagatctcaagatctggactactgttgaagaaattcccagtaaggctcacttatatctttagagatggcaaataactacaagaaaattgttctactgaaaggattagaggtcatcaatgattatcattttagaattgttaagtccttactgagtaacgatttaaaacttaatccaaaaatgaaagaagagtatgacaaaattcagattgctgacttgatggaggaaaagttcccaggtgatgccggtttgggcaaactaatagaattcttcaaagaaataccaacactgggagaccttgctgaaactcttaaaagagaaaagttaaaagtcaaaggaataatcccatctaaaaagacgaaacagaaagaagtgtatcctgctacacctgcatgcaccccaagcaaccgtctcacagctaaaggagcagaggagactcttggacctcagaaaagaaaaaaaccatctgaagaagagactggaaccaaaaggagtaagatgtccaaagagcagactcggccttcctgctctgcaggagccagcacgtccacagccatgggccgttccccacctccccagacctcatcatcagctccacccaacacttcctcaactgagagcctaaaaccattggccaaccgtcacgcaactgccagtaaaaatattttccgagaagacccaataatcgcgatggtactaaatgcaacaaaagtatttaaatatgaatcctcagaaaatgagcaaagaagaatgtttcatgctacagtggctacgcagacacagttctttcatgtgaaggttttaaacatcaacttgaagaggaaattcattaaaaagagaatcatcattatatcaaattattccaaacgtaatagtctcctagaggtgaatgaagcctcttctgtatctgaagctggtcctgaccaaacgtttgaggttccaaaggacatcatcagaagagcaaagaaaattccgaagatcaatattcttcacaaacaaacttcaggatatattgtatatggattatttatgctacatacgaaaattgtaaataggaagacgacaatctatgaaattcaggataaaacaggaagtatggctgtagtaggaaaaggagaatgccacaatatcccctgtgaaaaaggagataagcttcgactcttctgctttcgactgagaaagagggaaaatatgtcaaaactgatgtcagaaatgcatagtttcatccagatacagaaaaatacaaaccagagaagccatgactccaggagcatggcactaccccaggaacagagtcagcatccaaaaccttcagaggccagcacaaccctacctgaaagccatctcaagactcctcagatgccaccaacaaccccatccagcagttccttcaccaagaaggatgaaacccacccaggagcacagtcatcgcctgcaaactttagaatcacctcaccaactgtggcccctcctctttcttctgacacttccaccaaccgccatccagcagttccttaaataaggtcaccaaggacaaggatatcaaataac

SEQ ID NO: 3 - DNA, 1624 bp, Homo sapiens
aggaagccagcactagtcaagtgactctaagaagggccattttcccttgttgtctttgaaaatacttcattttcttagcatttcaggagattataacatcctgtatttcagtttctgagagctttactgactgatttccctattcaaaacaatcctcatttcctacatttctgaagatctcaagatctggactactgttgaagaaattcccagtaaggctcacttatatctttagagatggcaaataactacaagaaaattgttctactgaaaggattagaggtcatcaatgattatcattttagaattgttaagtccttactgagtaacgatttaaaacttaatccaaaaatgaaagaagagtatgacaaaattcagattgctgacttgatggaggaaaagttcccaggtgatgccggtttgggcaaactaatagaattcttcaaagaaataccaacactgggagaccttgctgaaactcttaaaagagaaaagttaaaagttgcaaataaaattgaatccattccagtcaaaggaataatcccatctaaaaagacgaaacagaaagaagtgtatcctgctacacctgcatgcaccccaagcaaccgtctcacagctaaaggagcagaggagactcttggacctcagaaaagaaaaaaaccatctgaagaagagactggaaccaaaaggagtaagatgtccaaagagcagactcggccttcctgctctgcaggagccagcacgtccacagccatgggccgttccccacctccccagacctcatcatcagctccacccaacacttcctcaactgagagcctaaaaccattggccaaccgtcacgcaactgccagtaaaaatattttccgagaagacccaataatcgcgatggtactaaatgcaacaaaagtatttaaatatgaatcctcagaaaatgagcaaagaagaatgtttcatgctacagtggctacgcagacacagttctttcatgtgaaggttttaaacatcaacttgaagaggaaattcattaaaaagagaatcatcattatatcaaattattccaaacgtaatagtctcctagaggtgaatgaagcctcttctgtatctgaagctggtcctgaccaaacgtttgaggttccaaaggacatcatcagaagagcaaagaaaattccgaagatcaatattcttcacaaacaaacttcaggatatattgtatatggattatttatgctacatacgaaaattgtaaataggaagacgacaatctatgaaattcaggataaaacaggaagtatggctgtagtaggaaaaggagaatgccacaatatcccctgtgaaaaaggagataagcttcgactcttctgctttcgactgagaaagagggaaaatatgtcaaaactgatgtcagaaatgcatagtttcatccagatacagaaaaatacaaaccagagaagccatgactccaggagcatggcactaccccaggaacagagtcagcatccaaaaccttcagaggccagcacaaccctacctgaaagccatctcaagactcctcagatgccaccaacaaccccatccagcagttccttcaccaaggtcaccaaggacaaggatatcaaataac

SEQ ID NO: 4 - DNA, 1597 bp, Homo sapiens
aggaagccagcactagtcaagtgactctaagaagggccattttcccttgttgtctttgaaaatacttcattttcttagcatttcaggagattataacatcctgtatttcagtttctgagagctttactgactgatttccctattcaaaacaatcctcatttcctacatttctgaagatctcaagatctggactactgttgaagaaattcccagtaaggctcacttatatctttagagatggcaaataactacaagaaaattgttctactgaaaggattagaggtcatcaatgattatcattttagaattgttaagtccttactgagtaacgatttaaaacttaatccaaaaatgaaagaagagtatgacaaaattcagattgctgacttgatggaggaaaagttcccaggtgatgccggtttgggcaaactaatagaattcttcaaagaaataccaacactgggagaccttgctgaaactcttaaaagagaaaagttaaaagtcaaaggaataatcccatctaaaaagacgaaacagaaagaagtgtatcctgctacacctgcatgcaccccaagcaaccgtctcacagctaaaggagcagaggagactcttggacctcagaaaagaaaaaaaccatctgaagaagagactggaaccaaaaggagtaagatgtccaaagagcagactcggccttcctgctctgcaggagccagcacgtccacagccatgggccgttccccacctccccagacctcatcatcagctccacccaacacttcctcaactgagagcctaaaaccattggccaaccgtcacgcaactgccagtaaaaatattttccgagaagacccaataatcgcgatggtactaaatgcaacaaaagtatttaaatatgaatcctcagaaaatgagcaaagaagaatgtttcatgctacagtggctacgcagacacagttctttcatgtgaaggttttaaacatcaacttgaagaggaaattcattaaaaagagaatcatcattatatcaaattattccaaacgtaatagtctcctagaggtgaatgaagcctcttctgtatctgaagctggtcctgaccaaacgtttgaggttccaaaggacatcatcagaagagcaaagaaaattccgaagatcaatattcttcacaaacaaacttcaggatatattgtatatggattatttatgctacatacgaaaattgtaaataggaagacgacaatctatgaaattcaggataaaacaggaagtatggctgtagtaggaaaaggagaatgccacaatatcccctgtgaaaaaggagataagcttcgactcttctgctttcgactgagaaagagggaaaatatgtcaaaactgatgtcagaaatgcatagtttcatccagatacagaaaaatacaaaccagagaagccatgactccaggagcatggcactaccccaggaacagagtcagcatccaaaaccttcagaggccagcacaaccctacctgaaagccatctcaagactcctcagatgccaccaacaaccccatccagcagttccttcaccaaggtcaccaaggacaaggatatcaaataac

SEQ ID NO: 5 - protein, 492 aa, Homo sapiens
MANNYKKIVLLKGLEVINDYHFRIVKSLLSNDLKLNPKMKEEYDKIQIADLMEEKFPGDAGLGKLIEFFKEIPTLGDLAETLKREKLKVANKIESIPVKGIIPSKKTKQKEVYPATPACTPSNRLTAKGAEETLGPQKRKKPSEEETGTKRSKMSKEQTRPSCSAGASTSTAMGRSPPPQTSSSAPPNTSSTESLKPLANRHATASKNIFREDPIIAMVLNATKVFKYESSENEQRRMFHATVATQTQFFHVKVLNINLKRKFIKKRIIIISNYSKRNSLLEVNEASSVSEAGPDQTFEVPKDIIRRAKKIPKINILHKQTSGYIVYGLFMLHTKIVNRKTTIYEIQDKTGSMAVVGKGECHNIPCEKGDKLRLFCFRLRKRENMSKLMSEMHSFIQIQKNTNQRSHDSRSMALPQEQSQHPKPSEASTTLPESHLKTPQMPPTTPSSSSFTKKDETHPGAQSSPANFRITSPTVAPPLSSDTSTNRHPAVP

SEQ ID NO: 6 - protein, 483 aa, Homo sapiens
MANNYKKIVLLKGLEVINDYHFRIVKSLLSNDLKLNPKMKEEYDKIQIADLMEEKFPGDAGLGKLIEFFKEIPTLGDLAETLKREKLKVKGIIPSKKTKQKEVYPATPACTPSNRLTAKGAEETLGPQKRKKPSEEETGTKRSKMSKEQTRPSCSAGASTSTAMGRSPPPQTSSSAPPNTSSTESLKPLANRHATASKNIFREDPIIAMVLNATKVFKYESSENEQRRMFHATVATQTQFFHVKVLNINLKRKFIKKRIIIISNYSKRNSLLEVNEASSVSEAGPDQTFEVPKDIIRRAKKIPKINILHKQTSGYIVYGLFMLHTKIVNRKTTIYEIQDKTGSMAVVGKGECHNIPCEKGDKLRLFCFRLRKRENMSKLMSEMHSFIQIQKNTNQRSHDSRSMALPQEQSQHPKPSEASTTLPESHLKTPQMPPTTPSSSSFTKKDETHPGAQSSPANFRITSPTVAPPLSSDTSTNRHPAVP

SEQ ID NO: 7 - protein, 461 aa, Homo sapiens
MANNYKKIVLLKGLEVINDYHFRIVKSLLSNDLKLNPKMKEEYDKIQIADLMEEKFPGDAGLGKLIEFFKEIPTLGDLAETLKREKLKVANKIESIPVKGIIPSKKTKQKEVYPATPACTPSNRLTAKGAEETLGPQKRKKPSEEETGTKRSKMSKEQTRPSCSAGASTSTAMGRSPPPQTSSSAPPNTSSTESLKPLANRHATASKNIFREDPIIAMVLNATKVFKYESSENEQRRMFHATVATQTQFFHVKVLNINLKRKFIKKRIIIISNYSKRNSLLEVNEASSVSEAGPDQTFEVPKDIIRRAKKIPKINILHKQTSGYIVYGLFMLHTKIVNRKTTIYEIQDKTGSMAVVGKGECHNIPCEKGDKLRLFCFRLRKRENMSKLMSEMHSFIQIQKNTNQRSHDSRSMALPQEQSQHPKPSEASTTLPESHLKTPQMPPTTPSSSSFTKVTKDKDIK

SEQ ID NO: 8 - protein, 452 aa, Homo sapiens
MANNYKKIVLLKGLEVINDYHFRIVKSLLSNDLKLNPKMKEEYDKIQIADLMEEKFPGDAGLGKLIEFFKEIPTLGDLAETLKREKLKVKGIIPSKKTKQKEVYPATPACTPSNRLTAKGAEETLGPQKRKKPSEEETGTKRSKMSKEQTRPSCSAGASTSTAMGRSPPPQTSSSAPPNTSSTESLKPLANRHATASKNIFREDPIIAMVLNATKVFKYESSENEQRRMFHATVATQTQFFHVKVLNINLKRKFIKKRIIIISNYSKRNSLLEVNEASSVSEAGPDQTFEVPKDIIRRAKKIPKINILHKQTSGYIVYGLFMLHTKIVNRKTTIYEIQDKTGSMAVVGKGECHNIPCEKGDKLRLFCFRLRKRENMSKLMSEMHSFIQIQKNTNQRSHDSRSMALPQEQSQHPKPSEASTTLPESHLKTPQMPPTTPSSSSFTKVTKDKDIK

SEQ ID NO: 9 - protein, 245 aa, Homo sapiens
MANNYKKIVLLKGLEVINDYHFRIVKSLLSNDLKLNPKMKEEYDKIQIADLMEEKFPGDAGLGKLIEFFKEIPTLGDLAETLKREKLKVANKIESIPVKGIIPSKKTKQKEVYPATPACTPSNRLTAKGAEETLGPQKRKKPSEEETGTKRSKMSKEQTRPSCSAGASTSTAMGRSPPPQTSSSAPPNTSSTEAYLMNLTLSLTAGSFLSCAVWGRDRELRNLELSATDSLSTAPIYYTPIPFAH

SEQ ID NO: 10 - DNA, 18 bp, Artificial Sequence
  Description of Artificial Sequence  Synthetic Primer
ggaacagagtcagcatcc

SEQ ID NO: 11 - DNA, 18 bp, Artificial Sequence
  Description of Artificial Sequence  Synthetic Primer
ctgctggatggcggttgg

SEQ ID NO: 12 - DNA, 20 bp, Artificial Sequence
  Description of Artificial Sequence  Synthetic Primer
tgaaggtcggagtcaacgga

SEQ ID NO: 13 - DNA, 20 bp, Artificial Sequence
  Description of Artificial Sequence  Synthetic Primer
ggcatggactgtggtcatga

SEQ ID NO: 14 - DNA, 19 bp, Artificial Sequence
  Description of Artificial Sequence  Synthetic Primer
agaccttgctgaaactctt

SEQ ID NO: 15 - protein, 21 aa, Homo sapiens
LKREKLKVANKIESIPVKGII

SEQ ID NO: 16 - protein, 12 aa, Homo sapiens
LKREKLKVKGII

SEQ ID NO: 17 - protein, 51 aa, Homo sapiens
PPTTPSSSSFTKKDETHPGAQSSPANFRITSPTVAPPLSSDTSTNRHPAVP

SEQ ID NO: 18 - protein, 20 aa, Homo sapiens
PPTTPSSSSFTKVTKDKDIK

SEQ ID NO: 19 - protein, 65 aa, Homo sapiens
TSSSAPPNTSSTESLKPLANRHATASKNIFREDPIIAMVLNATKVFKYESSENEQRRMFHATVAT

SEQ ID NO: 20 - protein, 65 aa, Homo sapiens
TSSSAPPNTSSTEAYLMNLTLSLTAGSFLSCAVWGRDRELRNLELSATDSLSTAPIYYTPIPFAH

SEQ ID NO: 21 - protein, 200 aa, Homo sapiens
MFHATVATQTQFFHVKVLNINLKRKFIKKRIIIISNYSKRNSLLEVNEASSVSEAGPDQTFEVPKDIIRRAKKIPKINILHKQTSGYIVYGLFMLHTKIVNRKTTIYEIQDKTGSMAVVGKGECHNIPCEKGDKLRLFCFRLRKRENMSKLMSEMHSFIQIQKNTNQRSHDSRSMALPQEQSQHPKPSEASTTLPESHLK

SEQ ID NO: 22 - protein, 6 aa, Homo sapiens
PQKRKK

SEQ ID NO: 23 - protein, 8 aa, Homo sapiens
LGPQKRKK